(19) United States
(12) Patent Application Publication
(10) Pub. No.: US 2026/0175034 A1
(43) Pub. Date: Jun. 25, 2026

(54) DETERMINATION OF CARDIAC PHYSIOLOGICAL CONDITIONS

(71) Applicant: Medtronic, Inc., Minneapolis, MN (US)

(72) Inventors: Hongyang Lu, Shanghai (CN); Tianyi Shi, Shanghai (CN); Robert Stadler, Shoreview, MN (US); Xiaohong Zhou, Woodbury, MN (US); Juan Meng, Shanghai (CN)

(21) Appl. No.: 19/129,785

(22) PCT Filed: Nov. 16, 2022
(86) PCT No.: PCT/CN2022/132220

§ 371 Date: May 14, 2025

(51) Int. Cl. A61N 1/368

(52) U.S. Cl. CPC A61N 1/36843

(57) ABSTRACT

A therapy system includes an implantable medical device including one or more implantable electrodes (40, 42, 48, 50, 94), and a computing apparatus (24) including processing circuitry and operably coupled to the one or more implantable electrodes (40, 42, 48, 50, 94). The one or more implantable electrodes (40, 42, 48, 50, 94) include a left bundle branch (LBB) electrode positionable adjacent a portion of a patient's LBB, and a right bundle branch (RBB) electrode positionable adjacent a portion of the patient's RBB. The computing apparatus (24) is configured to monitor electrical activity using the LBB and RBB electrodes, detect electrical activity based on the monitored electrical activity indicative of left ventricle (LV) depolarization and indicative of right ventricle (RV) depolarization, determine a temporal difference based on the detected electrical activity indicative of LV depolarization and the detected electrical activity indicative of RV depolarization, and determine whether there is a cardiac physiological condition based on the determined difference.

The present disclosure relates generally to determining one or more various cardiac physiological conditions using electrogram signal analysis to, for example, update or modify pacing parameters to optimize pacing delivered to a patient depending on the patient's needs. For example, pacing may be delivered to ensure capture of the cardiac conduction system as opposed to capture of myocardial tissues of the heart, or vice versa. More particularly, the present disclosure relates to electrogram signal analysis as opposed to electrocardiogram signal analysis, which, for example, may advantageously provide a more efficient means for determining cardiac physiological conditions, or may advantageously negate the need to travel to a clinic.

Implantable medical devices (IMDs), such as cardiac pacemakers or implantable cardioverter defibrillators, deliver therapeutic stimulation to patients' hearts thereby improving the lives of millions of patients living with heart conditions. Conventional pacing techniques involve pacing one or more of the four chambers of a patient's heart 12 as illustrated in FIG. 1, including the left atrium 33, the right atrium (RA) 26, the left ventricle 32 and the right ventricle 28. One common conventional therapeutic pacing technique that treats a slow heart rate, referred to as bradycardia, involves delivering an electrical pulse to a patient's right ventricular tissue. In response to the electrical pulse, both the right and left ventricles contract. However, the heartbeat process may be significantly delayed because the pulse travels from the right ventricle through the left ventricle. The electrical pulse passes through the muscle cells that are referred to as myocytes. Myocyte-to-myocyte conduction may be very slow. Delayed electrical pulses can cause the left ventricle to be unable to maintain synchrony with the right ventricle.

Over time, the left ventricle can become significantly inefficient at pumping blood to the body. In some patients, heart failure can develop such that the heart is too weak to pump blood to the body. Heart failure may be a devastating diagnosis since, for example, fifty percent of heart failure patients have a life expectancy of five years or less. Another possible cause of heart failure is due to atrial fibrillation, which is an irregular and often very rapid heart rhythm or arrhythmia. During atrial fibrillation, the atria of the heart can beat out of sync with the ventricles of the heart because of the arrythmia of the atria, which can lead to blood clots in the heart and increase the risk of stroke or heart failure, for example.

To avoid potential development of heart failure, some physicians have considered alternative pacing methods that involve the cardiac conduction system. Pacing the cardiac conduction system may quickly conduct electrical pulses (for example, akin to a car driving on a highway), whereas pacing cardiac muscle, or myocardial, tissue may more slowly conduct electrical pulses (for example, akin to a car driving on a dirt road).

The cardiac conduction system includes the sinoatrial node 1, atrial internodal tracts 2, 4, 5 (i.e., anterior internodal 2, middle internodal 4, and posterior internodal 5), atrioventricular node 3, His bundle 13 (also known as the atrioventricular bundle or bundle of His), left bundle branch 8a, and right bundle branch 8b as shown in FIG. 1. The arch of aorta 6 and the Bachman's bundle 7 are also shown in FIG. 1.

The sinoatrial node 1, located at the junction of the superior vena cava and right atrium, is considered to be the natural pacemaker of the heart as it continuously and repeatedly emits electrical impulses. The electrical impulses spread through the muscles of right atrium 26 to left atrium 33 to cause synchronous contraction of the atria. The electrical impulses are also carried through atrial internodal tracts to the atrioventricular node 3—the sole connection between the atria and the ventricles.

The conduction through the atrioventricular node or atrioventricular nodal tissue takes longer than through the atrial tissue, which results in a delay between the atrial contractions and the start of the ventricular contractions. The atrioventricular delay, which is the delay between atrial contractions and ventricular contractions, allows the atria to empty blood into the ventricles. Then, the valves between the atria and ventricles close in conjunction with ventricular contraction via branches of the bundle of His. The bundle of His, or His bundle, 13 is located in the membranous atrioventricular septum near the annulus of the tricuspid valve. The His bundle 13 splits into the left and right bundle branches 8a, 8b and are formed of specialized fibers called “Purkinje fibers” 9. The Purkinje fibers 9 may be described as being capable of rapidly conducting an action potential down the ventricular septum (VS), spreading the depolarization wavefront quickly through the remaining ventricular myocardium, and producing a coordinated contraction of the ventricular muscle mass.

Additionally, some patients may suffer from cardiac physiological conditions such as left bundle branch (LBB) or right bundle branch (RBB) block, where electrical impulses do not travel through the Purkinje fibers 9 normally. This can lead to un-coordinated contraction of the right and left ventricles. In such cases, for example, cardiac conduction system pacing therapy may require higher amplitude pacing pulses, or may require that pulses are delivered to the cardiac conduction system distal to (or farther along the cardiac conduction system than) the site of the LBB or RBB block, in the course of attempting to coordinate ventricular contraction. In some cases, it may be more optimal to pace the myocardial tissues of the heart, as opposed to the cardiac conduction system.

### SUMMARY

This disclosure generally relates to determining one or more various cardiac physiological conditions using electrogram (EGM) signal analysis, for example, to update or modify pacing modes and parameters to optimize pacing delivered to a patient depending on the patient's needs. LBB pacing is a physiological pacing approach, and LBB capture is required for such an approach. RBB pacing is a physiological pacing approach, and RBB capture is required for such an approach. In patients with LBB or RBB block, the electrical pulse may not propagate through the Purkinje fibers normally. In such cases, for examples, LBB or RBB pacing may require higher pulse outputs to break through the conduction block, or may pace the LBB or RBB at a location past, or distal to, the site of the block. Conversely, left ventricular (LV) or right ventricular (RV) septal pacing captures and paces at the respective septum, which is not part of the cardiac conduction system, and may provide another example of a pacing mode which may be used for patients with LBB or RBB block.

It can be difficult to implant a lead close enough to the LBB to effectively pace the LBB, or implanted LBB lead(s) may dislodge over time due to natural movement or due to injury, for example, and LV septal pacing may occur as a result. This is also true for the RBB pacing shifting into RV septal pacing. On one hand, for patients whose cardiac conduction systems work normally, septal pacing may be undesirable in some cases. On the other hand, for patients whose cardiac conduction systems do not work normally, septal pacing may be desirable in some cases, such as, for example, when the LBB or RBB block cannot be corrected or bypassed. In other cases for patients whose cardiac conduction systems do not work normally, cardiac conduction system pacing is still desirable, such as, for example, when the LBB or RBB block can be corrected or bypassed. Pacing modes may be chosen based on the individual patient's needs.

In particular, illustrative devices and methods are described herein to determine LBB or RBB function using EGM signal analysis (as opposed to ECG signal analysis with an external device) and to further determine various cardiac physiological conditions based on the EGM signal analysis and provide effective pacing therapy in response thereto. Use of EGM signals as opposed to ECG signals, for example, may advantageously provide more efficient or more effective analysis, provide timely modifications to the pacing parameters based on a patient's changed physiological conditions resulting in more effective pacing, and may negate the need for a patient to visit a clinic to have ECG signals measured. EGM signal analysis may indicate, for example, LBB block, RBB block, atrioventricular (AV) block, normal intrinsic conduction, and changes therebetween based on the difference between measured start of depolarization of the left and right ventricles.

In some examples, if the difference between the measured start of depolarization of the RV is before the measured start of depolarization of the LV, LBB block may be determined. If the difference between the measured start of depolarization of the LV is slightly before the measured start of depolarization of the RV (as determined using at least one threshold value), intrinsic cardiac conduction may be determined. If the measured start of depolarization of the LV is significantly before the measured start of depolarization of the RV (as determined using an RBB threshold value, which may or may not be the same value as the at least one threshold value described above), RBB block may be determined. AV block may be determined prior to any other determination, for example, by calculating the time from an atrial pace event to RV and LV depolarization, and if both depolarize at a time greater than an intrinsic AV conduction threshold, AV block may be determined.

Determination of various cardiac physiological conditions as described herein may be used to select and deliver a pacing configuration (e.g., cardiac conduction system pacing, myocardial pacing, or other pacing modes) that better serves the patient's needs, as described herein. For example, if a patient has LBB block, LBB pacing may need to be stronger (e.g., at a higher output voltage) to correct for the block, or the site of the block may need to be bypassed, or myocardial pacing of the LV may be more effective than LBB pacing to control LV depolarization and ensure ventricular synchrony. For another example, if a patient has RBB block, RBB pacing may need to be stronger to correct for the block, or the site of the block may need to be bypassed, or myocardial pacing of the RV may be more effective than RBB pacing to control RV depolarization and ensure ventricular synchrony. Additionally, selection of a pacing configuration to better serve the patient's needs may save battery life, prevent excessive pacing (which may cause, for example, cardiomyopathy or atrial fibrillation), and provide an automatic way to monitor and optimize cardiac pacing.

In one or more embodiments, illustrative devices and methods are described herein to periodically determine various cardiac physiological conditions, so as to provide effective cardiac therapy to a patient over time. Effective pacing modes may be desirable for a patient undergoing cardiac resynchronization therapy (CRT), for example. Further, such pacing modalities may need revisions over time with changing patient physiological conditions.

One illustrative therapy system may include an implantable medical device including a computing apparatus. The computer apparatus further includes processing circuitry and is operatively coupled to one or more implantable electrodes. The one or more implantable electrodes may include an LBB electrode positionable adjacent a portion of a patient's LBB, and an RBB electrode positionable adjacent a portion of the patient's RBB. The computing apparatus is configured to monitor electrical activity using the one or more implantable electrodes at the described locations. The computing apparatus is further configured to detect electrical activity based on the monitored electrical activity indicative of intrinsic LV depolarization and indicative of intrinsic RV depolarization. The computing apparatus is further configured to determine a temporal difference based on the detected electrical activity indicative of LV depolarization and the detected electrical activity indicative of RV depolarization. The computing apparatus is further configured to determine whether there is a cardiac physiological condition based on the determined difference.

One illustrative method may include monitoring electrical activity using one or more implantable electrodes comprising an LBB electrode positioned adjacent a portion of a patient's LBB and using an RBB electrode positioned adjacent a portion of the patient's RBB. The method may further include detecting electrical activity based on the monitored electrical activity indicative of LV depolarization and indicative of RV depolarization. The method may further include determining a difference based on the detected electrical activity indicative of LV depolarization and the detected electrical activity indicative of RV depolarization. The method may further include determining whether there is a cardiac physiological condition based on the determined difference.

One illustrative system may include a conduction system pacing lead. The conduction system pacing lead may include one or more implantable electrodes. The one or more implantable electrodes may include a left bundle branch (LBB) electrode positionable adjacent a portion of a patient's LBB and a right bundle branch (RBB) electrode positionable adjacent a portion of the patient's RBB. The system may further include an implantable medical device. The device may include a computing apparatus. The computing apparatus may include processing circuitry. The computing apparatus is operably coupled to the one or more implantable electrodes. The computing apparatus is configured to monitor electrical activity using the one or more implantable electrodes. The computing apparatus is further configured to detect electrical activity based on the monitored electrical activity indicative of LV depolarization and indicative of RV depolarization. The computing apparatus is further configured to determine a difference based on the detected electrical activity indicative of LV depolarization and the detected electrical activity indicative of RV depolarization. The computing apparatus is further configured to determine whether there is a cardiac physiological condition based on the determined difference.

The above summary is not intended to describe each embodiment or every implementation of the present disclosure. A more complete understanding will become apparent and appreciated by referring to the following detailed description and claims taken in conjunction with the accompanying drawings.

### BRIEF DESCRIPTION OF THE DRAWINGS

FIG. 1 is a schematic diagram of a heart and conduction system of a patient.

FIG. 2A is a conceptual diagram of an illustrative therapy system that is configured to provide cardiac conduction system pacing therapy to the left and/or right bundle branches using a lead placed in the right ventricle.

FIG. 2B is a detailed conceptual diagram showing the illustrative therapy system of FIG. 2A but only including two leads.

FIG. 3A is a conceptual diagram illustrating an illustrative therapy system that is configured to provide cardiac conduction system pacing therapy to the left bundle branch using a single lead placed in the right ventricle.

FIG. 3B is a close-up view of the lead in the patient's heart of FIG. 2A.

FIG. 4 is a functional block diagram illustrating an example of a configuration of an implantable medical device of FIGS. 2A-3B.

FIG. 5 is a block diagram of an illustrative method of determining cardiac physiological conditions based on monitored electrical activity that may be utilized by the devices of FIGS. 2-3, and optionally pacing the heart based on the determined cardiac physiological condition.

FIG. 6 is a block diagram of an illustrative method of determining a difference in the monitored electrical activity of FIG. 5.

FIG. 7 is a block diagram of an illustrative method of determining AV block illustrating determination of a cardiac physiological condition of the method of FIG. 5.

FIG. 8 is a block diagram of an illustrative method of determining LBB block illustrating determination of a cardiac physiological condition of the method of FIG. 5.

FIG. 9 is a block diagram of an illustrative method of determining RBB block illustrating determination of a cardiac physiological condition of the method of FIG. 5.

FIG. 10 is a block diagram of an illustrative method of determining no cardiac physiological condition of the method of FIG. 5.

FIG. 11 is a block diagram of another illustrative method of determining cardiac physiological conditions based on monitored electrical activity that may be utilized by the devices of FIGS. 2-3.

FIG. 12 is an illustrative diagram of a portion of a heart and a medical lead that may be utilized by the devices of FIGS. 2-3.

FIG. 13 is another depiction of exemplary ECG and EGM signals over time, illustrating determination of LBB block of the method of FIGS. 5 and 8.

FIG. 14 is another depiction of exemplary ECG and EGM signals over time, illustrating determination of RBB block of the method of FIGS. 5 and 9.

FIG. 15 is another depiction of exemplary ECG and EGM signals over time, illustrating determination of no cardiac physiological condition of the method of FIGS. 5 and 10.

### DETAILED DESCRIPTION

In the following detailed description of illustrative embodiments, reference is made to the accompanying figures of the drawing which form a part hereof, and in which are shown, by way of illustration, specific embodiments which may be practiced. It is to be understood that other embodiments may be utilized, and structural changes may be made without departing from (e.g., still falling within) the scope of the disclosure presented hereby.

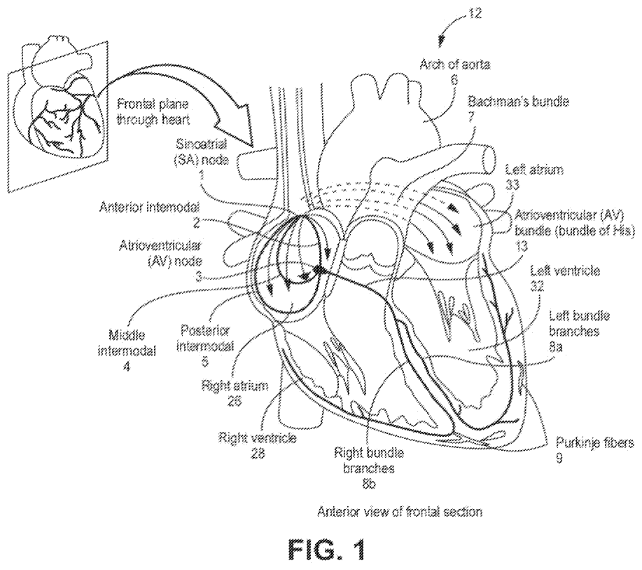

Illustrative systems, devices, and methods shall be described with reference to FIGS. 1-15. It will be apparent to one skilled in the art that elements or processes from one embodiment may be used in combination with elements or processes of the other embodiments, and that the possible embodiments of such systems, devices, and methods using combinations of features set forth herein is not limited to the specific embodiments shown in the Figures and/or described herein. Further, it will be recognized that the embodiments described herein may include many elements that are not necessarily shown to scale. Still further, it will be recognized that timing of the processes and the size and shape of various elements herein may be modified but still fall within the scope of the present disclosure, although certain timings, one or more shapes and/or sizes, or types of elements, may be advantageous over others.

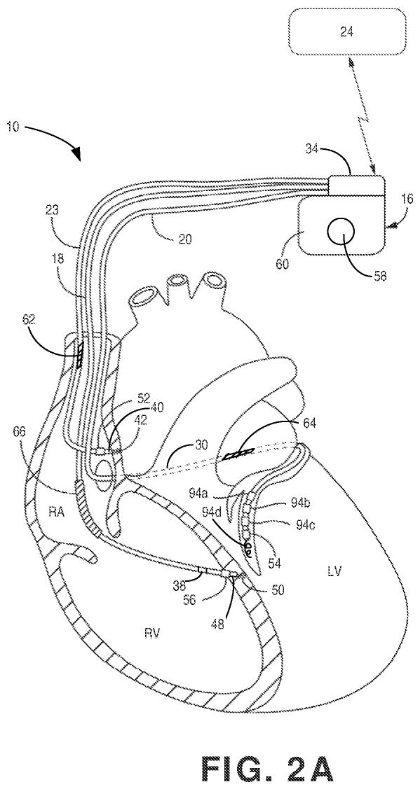

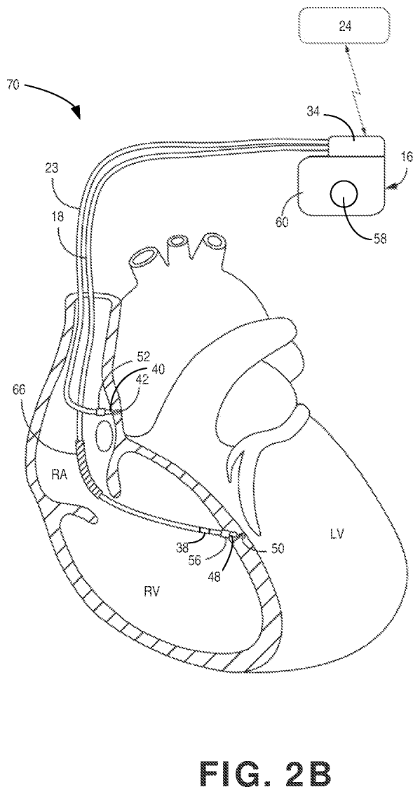

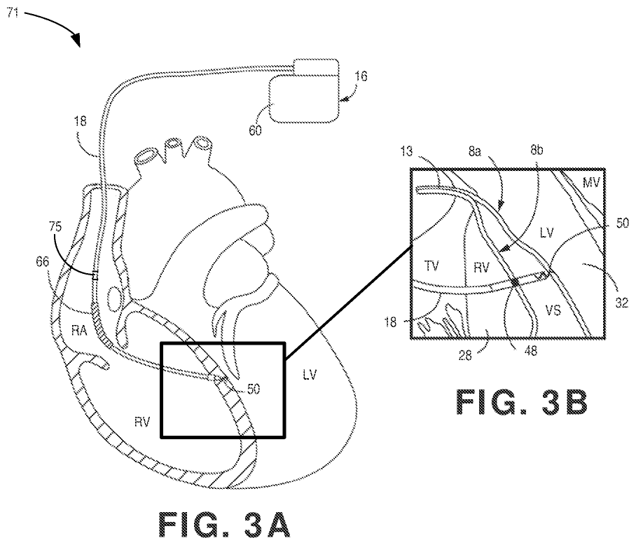

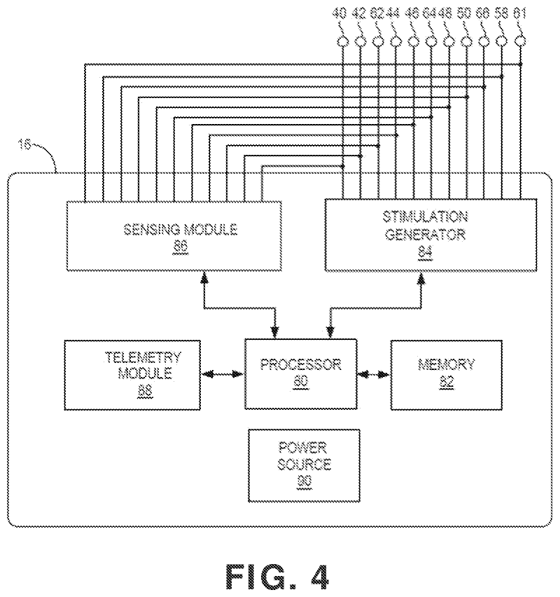

FIG. 1 depicts a schematic diagram of a heart 12 and FIGS. 2-3 depict conceptual diagrams showing illustrative therapy systems that may be used to provide therapy to the heart 12 of a patient. The patient ordinarily, but not necessarily, will be a human. As shown in FIGS. 2A-2B, the therapy system 10 may include IMD 16, which is coupled to three leads 18, 20, 23, and a programmer 24. The IMD 16 may be, for example, an implantable pacemaker, cardioverter, and/or defibrillator that provides electrical pulses to the heart 12 via electrodes coupled to one or more of the leads 18, 20, 23. Further non-limiting examples of the IMD 16 include the following: a pacemaker with a medical lead, an implantable cardioverter-defibrillator (ICD), an intracardiac device, a leadless pacing device (LPD), a subcutaneous ICD (S-ICD), and a subcutaneous medical device (e.g., nerve stimulator, inserted monitoring device, etc.).

The leads 18, 20, 23 may extend into the heart 12 of the patient to sense electrical activity of the heart 12 and/or deliver electrical stimulation to the heart 12. In the example shown in FIG. 2A, the right atrial (RA) lead 23 extends through one or more veins (not shown), the superior vena cava (not shown), and into the right atrium 26. The right atrial lead 23 may be positioned for positioning electrodes 40, 42 near, adjacent, on, within, or around the right atrium for sensing electrocardiogram signals and pacing the right atrial myocardium. The RA lead 23 is shown with a ring electrode 40 and a helix tip electrode 42 that may be selected in various bipolar pacing electrode pairs for pacing the right atrial myocardial tissue and for sensing right atrial epicardial electrocardiogram signals. One of the electrodes 40, 42 may be selected in combination with IMD housing 60 or a coil electrode (62) for delivering unipolar right atrial myocardial pacing and/or sensing unipolar atrial electrocardiogram signals.

The left ventricular coronary sinus lead 20 extends through one or more veins, the vena cava, the right atrium 26, and into the coronary sinus 30 to a region adjacent to the free wall of the left ventricle 32 of the heart 12. The left ventricular coronary sinus lead 20 may be positioned for positioning electrodes 94a, 94b, 94c, 94d (collectively “94”) epicardially along the left ventricular myocardium for sensing electrocardiogram signals and pacing the left ventricular myocardium. The left ventricular coronary sinus lead 20 is shown as a quadripolar lead carrying four electrodes 94a-94d that may be selected in various bipolar pacing electrode pairs for pacing the left ventricular myocardial tissue and for sensing left ventricular epicardial electrocardiogram signals. One of the electrodes 94 may be selected in combination with IMD housing 60 or a coil electrode (64) for delivering unipolar left ventricular myocardial pacing and/or sensing unipolar ventricular electrocardiogram signals.

In one embodiment, the cardiac conduction system pacing therapy lead 18 (e.g., left bundle branch pacing lead, right bundle branch pacing lead, His-bundle pacing lead, etc.) extends through one or more veins and the vena cava, the right atrium 26, through the tricuspid valve and into the right ventricle 28 of the heart 12 to pace the cardiac conduction system (e.g., within the ventricular septal wall, proximate and/or in direct contact with the left bundle branch 8a, proximate and/or in direct contact with the right bundle branch 8b, proximate and/or in direct contact with the His bundle 13, etc.). In some embodiments, the cardiac conduction system pacing therapy lead 18 may be positioned within about 1 millimeter of a portion of the cardiac conduction system such as, e.g., the His bundle 13, the left bundle branch 8a, the right bundle branch 8b, etc. The cardiac conduction system pacing therapy lead 18 may be positioned for positioning electrodes 48, 50 near, adjacent, on, within, or around the RBB, LBB (respectively) for sensing electrocardiogram signals and pacing the cardiac conduction system. The cardiac conduction system pacing therapy lead 18 is shown with a ring electrode 48 and a helix tip electrode 50 that may be selected in various bipolar pacing electrode pairs for pacing the RBB and the LBB (respectively) and for sensing RBB and LBB electrocardiogram signals (respectively). One of the electrodes 48, 50 may be selected in combination with IMD housing 60 or a coil electrode (66) for delivering unipolar RBB and LBB pacing and/or sensing unipolar RBB and LBB electrocardiogram signals. In alternative embodiments, the cardiac conduction system pacing therapy lead 18 is also used to pace the RA using an electrode 75 (shown in FIGS. 3A-B), or is used to pace the RA in addition to the cardiac conduction system, such as in a two lead configuration which does not include the RA lead 23.

One example of a cardiac conduction system pacing therapy lead (e.g., a His lead) can be the SELECTSECURE™ 3830. A description of the SELECTSECURE™ 3830 is found in the Medtronic model SELECTSECURE™ 3830 manual (2013), incorporated herein by reference in its entirety. The SELECTSECURE™ 3830 includes two conductors without lumens.

As used herein, cardiac conduction system pacing therapy refers to any techniques that are configured to deliver pacing therapy (e.g., pacing pulses, electrical stimulation, etc.) to the cardiac conduction system including, e.g., the His bundle 13, the left bundle branch 8a, the right bundle branch 8b, etc., in order to initiate activation. As used herein, the term “activation” refers to a sensed or paced event. For example, an atrial activation may refer to an atrial sense or event (As) or an atrial pace or artifact of atrial pacing (Ap). As will be described herein, an atrial sense may be detected, or identified, in one or more various signals monitored using one or more various devices or sensors located in one or more various locations. For example, an atrial sense may be detected in a near-field electrical signal using an electrode positioned in the right atrium with a respective reference electrode (e.g., an electrode on the housing of the implantable medical device). Further, for example, an atrial sense may be detected in a far-field electrical signal using electrodes positioned outside of the right atrium such as in the right ventricle or ventricular septum and a respective reference electrode. Still, for example, an atrial sense may be detected in a far-field signal using a mechanical cardiac activation sensor such as an accelerometer or microphone (e.g., a heart sound sensor) positioned outside of the right atrium such as in the right ventricle or ventricular septum or another portion of the patient's body (e.g., within the can or housing of an IMD positioned outside of the patient's heart). Similarly, a ventricular activation may refer to a ventricular sense or event (Vs) or a ventricular pace or artifact of ventricular pacing (Vp), which may be described as ventricular stimulation pulses. In some embodiments, an activation interval can be detected from As or Ap to Vs or Vp, as well as Vp to Vs. In particular, activation intervals may include a pacing (Ap or Vp) to ventricular interval (left ventricular or right ventricular sense) or an atrial-sensing (As) to ventricular-sensing interval (left ventricular or right ventricular).

Illustrative IMDs may be described as delivering one or both of conventional pacing therapy and cardiac conduction system pacing therapy. Conventional, or traditional, pacing therapy may be described as delivering pacing pulses into myocardial tissue that is not part of the cardiac conduction system of the patient's heart such that, e.g., the pacing pulses trigger electrical activation that propagates primarily from one myocardial cell to another myocardial cell (also referred to as “cell-to-cell”) as opposed to propagating within the cardiac conduction system prior to the myocardial tissue. For instance, conventional pacing therapy may deliver pacing pulses directly into the muscular heart tissue (e.g., myocardial tissue) that is to be depolarized to provide the contraction of the heart. For example, conventional left ventricular pacing therapy may utilize a left ventricular coronary sinus lead 20 that is implanted so as to extend through one or more veins, the vena cava, the right atrium 26, and into the coronary sinus 30 to a region adjacent to the free wall of the left ventricle 32 of the heart 12 so as to deliver pacing pulses to the myocardial tissue of the free wall of the left ventricle 32.

An illustrative left ventricular lead 20 with a set of spaced apart electrodes is shown in U.S. Pat. Pub. No. WO 2019/104174 A1, filed on May 4, 2012, by Ghosh et al., which is incorporated by reference in its entirety herein. Illustrative electrodes on leads to form pacing vectors are shown and described in U.S. Pat. No. 8,355,784 B2, and U.S. Pat. No. 8,126,546, each of which are incorporated by reference in their entireties.

Additionally, the pacing therapy leads 18, 20, 23 may be utilized to deliver left ventricle or left ventricular septal pacing to the ventricular septal wall. At least one of pacing therapy leads 18, 20, 23 may extend through one or more veins, the vena cava, right atrium 26, and into the coronary sinus 30 to a region adjacent to the septal wall of left ventricle 32 of heart 12.

Illustrative cardiac conduction system pacing therapy may be described in, for example, U.S. Pat. App. Pub. No. 2019/0111270 A1 entitled “His Bundle and Bundle Branch Pacing Adjustment” published on Apr. 18, 2019, which is incorporated herein by reference in its entirety. Illustrative left ventricular septal pacing may be described in, for example, U.S. patent application Ser. No. 16/521,000 entitled “AV Synchronous Septal Pacing” filed on Jul. 24, 2019, which is incorporated herein by reference in its entirety.

One or more elongated conductors of any of the leads 18, 20, 23 may extend through a hermetic feedthrough assembly, and within an insulative tubular member of the respective lead, and may electrically couple an electrical pulse generator (contained within housing) to one or more electrodes such as, e.g., ring electrodes, tips electrodes, helical electrodes, etc. The conductors may be formed by one or more electrically conductive wires comprising, for example, MP35N alloy known to those skilled in the art, in a coiled or cabled configuration, and the insulative tubular member may be any suitable medical grade polymer, for example, polyurethane, silicone rubber, or a blend thereof. According to one or more illustrative embodiments, the flexible lead body may extend a pre-specified length (e.g., about 10 centimeters (cm) to about 20 cm, or about 15 to 20 cm) from a proximal end to a distal end. The lead body may be less than about 7 French (FR) but typically in the range of about 3 FR to 4 FR in size. In one or more embodiments, about 2 FR size to about 3 FR size lead body is employed.

Cardiac conduction system pacing may include at least one of His bundle pacing, LBB pacing, and RBB pacing. Bundle branch pacing may bypass the pathological region and may have a low and stable pacing threshold. In some embodiments, only one of the left bundle branch or the right bundle branch may be paced using one or more pacing leads. In further embodiments, both bundle branches may be paced at the same time (e.g., dual bundle branch pacing), which may mimic intrinsic activation propagation via the His bundle-Purkinje conduction system, e.g., paced activation propagates via both bundle branches to both ventricles for synchronized contraction. His bundle pacing, on the other hand, typically paces the His bundle proximal to the bundle branches. In some embodiments, the IMD 16 may be coupled to one, two, or more electrodes located in one or more bundle branches configured for bundle branch pacing.

In some embodiments, the IMD 16 may be an intracardiac pacemaker or leadless pacing device (LPD) configured to pace one or more portions of the cardiac conduction system such as one or both of the bundle branches. As used herein, “leadless” refers to a device being free of a lead extending out of the heart 12. In other words, a leadless device may have a lead that does not extend from outside of the heart to inside of the heart. Some leadless devices may be introduced through a vein, but once implanted, the leadless devices are free of, or may not include, any transvenous lead and may be configured to provide cardiac therapy without using any transvenous lead. In one or more embodiments, an illustrative LPD for bundle pacing does not use a lead to operably connect to an electrode disposed proximate to the septum when a housing of the device is positioned in the atrium. A leadless electrode may be leadlessly coupled to the housing of the medical device without using a lead between the electrode and the housing.

The IMD 16 may sense electrical signals attendant to the depolarization and repolarization of the heart 12 via various electrodes as shown in FIG. 2A coupled to at least one of leads 18, 20, 23. In some examples, the IMD 16 provides pacing pulses to the heart 12 based on the electrical signals sensed within the heart 12. The configurations of the electrodes used by the IMD 16 for sensing and pacing may be unipolar or bipolar.

The IMD 16 may also provide defibrillation therapy and/or cardioversion therapy via electrodes located on at least one of the leads 18, 20, 23. For example, the IMD 16 may detect atrial arrhythmias of heart 12, such as atrial fibrillation of the atria 26, 33, and then may deliver defibrillation therapy to the heart 12 in the form of electrical pulses. Also, the IMD 16 may detect ventricular arrhythmias of the heart 12, such as ventricular fibrillation of the ventricles 28, 32, and then may deliver defibrillation therapy to the heart 12 in the form of electrical pulses. In some examples, the IMD 16 may be programmed to deliver a progression of therapies, e.g., pulses with increasing energy levels, until fibrillation of the heart 12 is stopped. The IMD 16 may detect fibrillation employing one or more fibrillation detection techniques known in the art.

In some examples, the programmer 24 as shown in FIGS. 2A-B may be a handheld computing device or a computer workstation or a mobile phone. The programmer 24 may include a user interface that receives input from a user. The user interface may include, for example, a keypad and a display, which may for example, be a cathode ray tube (CRT) display, a liquid crystal display (LCD) or light emitting diode (LED) display. The keypad may take the form of an alphanumeric keypad, or a reduced set of keys associated with particular functions. The programmer 24 can additionally or alternatively include a peripheral pointing device, such as a mouse, via which a user may interact with the user interface. In some embodiments, a display of the programmer 24 may include a touch screen display, and a user may interact with the programmer 24 via the display. Through the graphical user interface on the programmer 24, a user may configure one or more pacing therapies, select one or more pacing modes, etc.

Additionally, various pacing settings may be adjusted, or configured, based on various sensed signals. For example, various near-field and far-field signals may be sensed by one or more of the electrodes coupled to the IMD 16 and/or other devices operatively coupled thereto. For example, right ventricular depolarization and left ventricular depolarization intervals may be monitored or measured within a near-field or far-field signal and then may be used to adjust, configure, and select cardiac conduction system pacing therapy. Further, for example, QRS morphology (e.g., QRS peak, various QRS intervals) may be monitored or measured within a near-field or far-field signal and then may be used to adjust, configure, and select cardiac conduction system pacing therapy. Still further, for example, one or more of right ventricular depolarization and left ventricular depolarization interval consistency, and QRS morphology consistency may be monitored or measured within a near-field or far-field signal and then may be used to adjust, configure, and select cardiac conduction system pacing therapy.

The illustrative therapy systems described herein such as IMD 16 may be utilized to deliver cardiac conduction system pacing therapy according to a variety of different modes such as, e.g., inhibited pacing mode, ventricular fusion pacing mode, atrioventricular synchronous pacing mode, atrial fibrillation pacing mode, etc.

The ventricular fusion pacing mode may be configured to deliver cardiac conduction system pacing therapy to provide effective ventricular fusion. Effective ventricular fusion may be described as synchronizing the timing of the left ventricular activation with the activation on the right ventricle. For example, in a fusion pacing configuration, a medical device may deliver one or more pacing pulses in order to pre-excite the left ventricle and synchronize the depolarization of the left ventricle with the depolarization of the earlier contracting right ventricle. The ventricular activation of the left ventricle may “fuse” (or “merge”) with the ventricular activation of the right ventricle that is attributable to intrinsic conduction of the heart. In this way, the intrinsic and pacing-induced excitation wave fronts may fuse together such that the depolarization of the left ventricle is resynchronized with the depolarization of the right ventricle.

For a patient experiencing LBB block, the selected pacing mode (e.g., inhibited pacing mode, ventricular fusion pacing mode, etc.) may be further configured to provide LBB pacing pulses at a high enough voltage output to correct the LBB block, or may be configured to provide LBB pacing pulses at a location distal to the block such that the block is bypassed, or may be configured to stop any ongoing delivery of cardiac conduction system pacing through the LBB and deliver left ventricular septal pacing using an implantable electrode. For a patient experiencing RBB block, the selected pacing mode (e.g., inhibited pacing mode, ventricular fusion pacing mode, etc.) may be further configured to provide RBB pacing pulses at a high enough voltage output to correct the RBB block, or may be configured to provide RBB pacing pulses at a location distal to the block such that the block is bypassed, or may be configured to stop any ongoing delivery of cardiac conduction system pacing through the RBB and deliver right ventricular septal pacing using an implantable electrode. These effectively switch the mode from cardiac conduction system pacing to more traditional myocardial pacing, in an effort to achieve ventricular fusion.

As used herein, the term “far-field” electrical signal refers to the result of measuring cardiac activity using a sensor, such as an electrode, positioned outside of an area of interest. For example, a far-field electrical signal representing electrical activity of a chamber of interest of the patient's heart may be measured from an electrode positioned in an adjacent chamber (i.e., a chamber different from than that of the chamber of interest that is next to or near the chamber of interest). More specifically, for example, atrial electrical activity, or electrical activity originating one or more both atria, representative of depolarization of the one or both atria may be monitored in a far-field electrical signal measured using an electrode positioned outside of the right atrium such as in the right or left ventricle, or in the ventricular septum. As used herein, the term “near-field” electrical signal refers to the result of measuring cardiac activity using a sensor, such as an electrode, positioned near an area of interest. For example, an electrical signal measured using an electrode positioned on the left side of the patient's ventricular septum is one example of a near-field electrical signal of the patient's LV.

P-wave timing is the time at which a P-wave is detected. Typically, P-wave timing includes using the maximal first derivative of a P-wave upstroke (or the time of the maximal P-wave value). P-wave timing is also used in the device marker channel to indicate the time of the P-wave or the time of atrial activation. P-wave timing may be determined using near-field signals obtained by sensors (e.g., electrodes, accelerometers, heart sound sensors, etc.) positioned in the atria (e.g., the right atrium) and/or far-field near-field signals obtained by sensors (e.g., electrodes, accelerometers, heart sound sensors, etc.) positioned outside of the atria (e.g., the right atrium) such as in the right ventricle and/or ventricular septum.

R-wave timing is the time at which the QRS complex is detected. Typically, R-wave timing includes using the maximal first derivative of an R-wave upstroke (or the time of the maximal R-wave value). R-wave timing is also used in the device marker channel to indicate the time of the R-wave or the time of ventricular activation.

A user, such as a physician, technician, or other clinician, may interact with the programmer 24 to communicate with the IMD 16. For example, the user may interact with the programmer 24 to retrieve physiological or diagnostic information from the IMD 16. Additionally, a user may also interact with the programmer 24 to program the IMD 16, e.g., select values for operational parameters of the IMD 16. The IMD 16 and programmer 24 may communicate via wireless communication using any techniques known in the art. Examples of communication techniques may include, for example, low frequency or radiofrequency (RF) telemetry, but other techniques are also contemplated. In some examples, the programmer 24 may include a programming head that may be placed proximate to the patient's body near the IMD 16 implant site in order to improve the quality or security of communication between the IMD 16 and the programmer 24.

The three chamber IMD 16 may be used for cardiac resynchronization therapy and defibrillation or cardioversion therapy (CRT-D). The leads 18, 20, 23 may be electrically coupled to a stimulation generator, a sensing module, or other modules of IMD 16 via connector block 34. In some examples, proximal ends of leads 18, 20, 23 may include electrical contacts that electrically couple to respective electrical contacts within the connector block 34. In addition, in some examples, the leads 18, 20, 23 may be mechanically coupled to the connector block 34 with the aid of set screws, connection pins, or another suitable mechanical coupling mechanism.

While the cardiac conduction system pacing therapy lead 18 is shown and described with respect to FIGS. 2A, 2B, 3A, and 3B as being placed in the RV along the intraventricular septal wall, in other examples, the cardiac conduction system pacing therapy lead 18 may be placed in the right atrium within the triangle of Koch region (not shown) with the corresponding electrodes 48, 50 tunneled through the septal tissue to be positioned proximate the RBB and LBB, respectively. In such examples, the system may not contain a lead positioned within the RV, yet still obtain the benefit of LBB or RBB pacing and sensing as described herein. Additionally or alternatively, the system in such examples may include an additional lead or electrode(s) (e.g., RA lead 23 or electrode(s) 40, 42) positioned in the RA configured to pace the RA that may be different from the cardiac conduction system pacing therapy lead 18 or the respective LBB and RBB electrodes.

Each of the leads 18, 20, 23 includes an elongated, insulative lead body, which may carry any number of conductors. In the illustrated example, an optional pressure sensor 38 and bipolar electrodes 48 and 50 are located proximate to a distal end of the cardiac conduction system pacing therapy lead 18. The pressure sensor 38 may respond to an absolute pressure inside RV, or may be positioned within other regions of the heart 12 or elsewhere within or proximate to the cardiovascular system of the patient to monitor cardiovascular pressure associated with mechanical contraction of the heart. In addition, in some examples, the pressure sensor 38 may be self-contained device that is implanted within the heart 12 and wirelessly correspond with the IMD 16. In addition, the bipolar electrodes 94a-94d (collectively referred to as reference number 94) are located proximate to a distal end of the left ventricular lead 20 and bipolar electrodes 40 and 42 are located proximate to a distal end of RA lead 23. The electrodes 48, 50 may be used for pacing and/or sensing of the cardiac conduction system tissue (e.g., His bundle or bundle branch tissue).

The electrodes 40, 94a-c and 48 may take the form of ring electrodes, and the electrodes 42, 94d and 50 may take the form of extendable and/or fixed helix tip electrodes mounted within the insulative electrode heads 52, 54 and 56, respectively. Each of the electrodes 40, 42, 94, 48 and 50 may be electrically coupled to a respective one of the coiled conductors within the lead body of its associated lead 23, 20, 18, and thereby coupled to respective ones of the electrical contacts on the proximal end of the leads 23, 20, 18.

The electrodes 40, 42, 94, 48 and 50 may sense electrical signals attendant to the depolarization and repolarization of the heart 12. The electrical signals are conducted to the IMD 16 via the respective leads 23, 20, 18. In some examples, the IMD 16 also delivers pacing pulses via the electrodes 40, 42, 94, 48, 50 to cause depolarization of cardiac tissue of heart 12. In some examples, as illustrated in FIG. 2A, the IMD 16 may include one or more housing electrodes, such as housing electrode 58, which may be formed integrally with an outer surface of a hermetically sealed housing 60 of the IMD 16 or otherwise coupled to the housing 60. In some examples, the housing electrode 58 may be defined by an uninsulated portion of an outward facing portion of the housing 60 of the IMD 16. Other divisions between insulated and uninsulated portions of housing 60 may be employed to define two or more housing electrodes. In some examples, the housing electrode 58 includes substantially all of the housing 60. Any of the electrodes 40, 42, 94, 48, 50 may be used for unipolar sensing or pacing in combination with the housing electrode 58 or for bipolar sensing with two electrodes in the same pacing lead. In one or more embodiments, the housing 60 may enclose a stimulation generator (see FIG. 4) that generates cardiac pacing pulses and defibrillation or cardioversion shocks, as well as a sensing module for monitoring the patient's heart rhythm.

The leads 23, 20, 18 may also include elongated electrodes 62, 64, 66, respectively (shown in FIG. 2A), which may take the form of a coil. The IMD 16 may deliver defibrillation shocks to the heart 12 via any combination of the elongated electrodes 62, 64, 66, and the housing electrode 58. The electrodes 58, 62, 64, 66 may also be used to deliver cardioversion pulses to the heart 12. The electrodes 62, 64, 66 may be fabricated from any suitable electrically conductive material, such as, but not limited to, platinum, platinum alloy or other materials known to be usable in implantable defibrillation electrodes.

The elongated electrodes may be selected in a unipolar electrode vector with any of the lead-based tip or ring electrodes for sensing unipolar electrocardiogram signals for analysis and determination of ventricular conduction conditions. In some instances, the elongated electrodes may be used with the housing 60 for sensing a far-field electrocardiogram signal for use in determining atrial depolarizations or activations, etc. The pressure sensor 38 may be coupled to one or more coiled conductors within the lead 18.

FIGS. 2B-3B are conceptual diagrams illustrating additional examples of a dual chamber therapy system 70 and a single chamber therapy system 71. The therapy system 70 is similar to therapy system 10 of FIG. 2A, but includes two leads 18, 23, rather than three leads. The therapy system 70 may utilize the IMD 16 configured to deliver, or perform, dual chamber pacing. The leads 18, 23 are implanted within the RV and the RA to pace one or more portions of the cardiac conduction system such as the His bundle or one or both bundle branches, and to pace the RA, respectively. The therapy system 71 is similar to therapy system 10 of FIG. 2A, but includes a single cardiac conduction system pacing lead 18, rather than three leads. The therapy system 71 may utilize the IMD 16 configured to deliver, or perform, single chamber pacing. The cardiac conduction system pacing lead 18 is implanted the RV to pace one or more portions of the cardiac conduction system such as one or both bundle branches.

The cardiac conduction system pacing lead 18 may be include an electrode 50 in the form of a helix (also referred to as a helical electrode) that may be positioned proximate to, near, adjacent to, or in, area or portions of the cardiac conduction system such as, e.g., ventricular septum, triangle of Koch, the His bundle, left bundle branch tissues, and/or right bundle branch tissue. The cardiac conduction system pacing lead 18 may be configured as a bipolar lead that may be used with a pacemaker device, a CRT-P device, or a CRT-ICD.

FIGS. 3A-3B show the patient's heart 12 implanted with cardiac conduction system pacing lead 18 to deliver bundle branch pacing according to one example of the single chamber therapy system 71. The cardiac conduction system therapy lead 18 is positioned, or located, through the tricuspid valve into the RV and implanted in the interventricular septum, e.g., about 1 to 2 centimeters in an apical direction away from the RA (as illustrated in FIGS. 2A-B). FIG. 3B is a close-up view of the cardiac conduction system therapy lead 18 in the patient's heart 12 of FIG. 3A. In some embodiments, the cardiac conduction system therapy lead 18 may be the only lead implanted in the heart 12. In other embodiments as discussed herein, there may be leads in addition to the cardiac conduction system therapy lead 18 implanted in the heart 12. The one or more implantable electrodes of the cardiac conduction system therapy lead 18 may include a pacing electrode implantable proximate the cardiac conduction system to deliver cardiac conduction system pacing therapy.

As illustrated, the cardiac conduction system pacing therapy lead 18 is implanted in the septal wall, or ventricular septum, from the RV toward the LV. The cardiac conduction system pacing therapy lead 18 may not pierce through the wall of the LV or extend into the LV chamber. The electrodes 48 and 50 may be disposed on a distal end portion of the cardiac conduction system pacing therapy lead 18 as discussed herein at least with respect to FIG. 2A. The cardiac conduction system pacing therapy lead 18 may also be described as a shaft. The electrodes 48 and 50 may be the same as or similar to electrode 48 and electrode 50 shown in FIG. 2A and the electrode 48 is configured to sense or pace the right bundle branch and the electrode 50 is configured to sense or pace the left bundle branch, for example, during dual bundle branch pacing. Accordingly, the electrode 48 may be implanted near right bundle branch 8b, and the electrode 50 may be implanted near the left bundle branch 8a. The electrode 50 may be implanted towards the left side of the patient's ventricular septum. The electrode 48 may be implanted towards the right side of the patient's ventricular septum. In one embodiment, the electrode 50 may be a helix electrode, and the electrode 48 may be a ring electrode.

During dual bundle branch pacing, both the electrodes 48 and 50 may each deliver a pulse to achieve synchronized activation, or excitation, of the right bundle branch 8b and the left bundle branch 8a, which may result in synchronized activation of the RV and the LV. In some embodiments, the pulses may be delivered at the same time to achieve synchrony. In other embodiments, the pulses may be delivered with a delay to achieve synchrony.

Although the cardiac conduction system pacing therapy lead 18 as shown in configured for dual bundle branch pacing using the electrodes 48, 50, it is to be understood that the cardiac conduction system pacing therapy lead 18 or leads similar thereto are considered herein that may only include one of the electrode 48 and the electrode 50, and thus, only configured to deliver cardiac conduction system pacing therapy to one of the right bundle branch and the left bundle branch. In alternative embodiments, both electrodes 48 and 50 may be located on the cardiac conduction system pacing therapy lead 18, but the IMD 16 may use just one of electrodes 48, 50 to pace only one bundle branch.

Additionally, the cardiac conduction system pacing therapy lead 18 may include an RA electrode 75 disposed more proximal to the electrodes 48, 50 along the cardiac conduction system pacing therapy lead 18. The RA electrode 75 may be positioned in or near the RA and may function as an anode for cathodal pulses from the electrode 48 and/or the electrode 50. Further, the RA electrode 75 may provide atrial sensing to, e.g., sense atrial depolarizations or activations, to sense or detect atrial fibrillation, etc. Although the cardiac conduction system pacing therapy lead 18 as shown includes the RA electrode 75, it is to be understood that the cardiac conduction system pacing therapy lead 18 may not include the RA electrode 75, and instead, only include one or both of the electrode 48 and the electrode 50.

When the cardiac conduction system pacing therapy lead 18 is positioned for delivering bundle branch pacing, of one or both bundle branches, cardiac conduction system pacing therapy may be combined with traditional ventricular myocardial pacing of the left ventricle using the coronary sinus lead 20 to correct a left ventricular conduction delay and achieve electrical and mechanical synchrony of the left and right ventricles. As such, in some examples, one or more processors, one or more processing circuits, or a computing apparatus of the IMD 16 may select a cardiac conduction system pacing therapy plus traditional left ventricular myocardial pacing therapy that includes, for example, single or bilateral bundle branch pacing, e.g., using the cardiac conduction system pacing therapy lead 18, combined with left ventricular myocardial pacing using the coronary sinus lead 20.

The configuration of therapy systems 10, 70, and 71 illustrated in FIGS. 2-3 are merely examples. In other examples, a therapy system may include epicardial leads and/or patch electrodes instead of or in addition to the transvenous leads 18, 20, 23 illustrated in FIGS. 2-3 or other configurations shown or described herein or incorporated by reference. Further, the IMD16 need not be implanted within patient. As such, it is to be understood that the illustrative therapy systems described herein may include any suitable number of leads coupled to IMD 16, and each of the leads may extend to any location within or proximate to the heart 12. For example, illustrative therapy systems may include three transvenous leads located as illustrated in FIG. 2A, a single transvenous lead located as illustrated in FIGS. 3A-3B, or two transvenous leads located as illustrated in FIG. 2B.

FIG. 4 is a functional block diagram of one example configuration of the IMD 16. The IMD 16 of FIG. 4 may be substantially similar to the IMD 16. The IMD 16 includes a processor 80, a memory 82, a stimulation generator 84 (e.g., electrical pulse generator or signal generating circuit), a sensing module 86 (e.g., sensing circuit), a telemetry module 88, and a power source 90. One or more components of the IMD 16, such as the processor 80, may be contained within a housing of the IMD 16 (e.g., within a housing of a pacemaker). The telemetry module 88, the sensing module 86, or both the telemetry module 88 and the sensing module 86 may be included in a communication interface. The memory 82 includes computer-readable instructions that, when executed by the processor 80, cause the IMD 16 and the processor 80 to perform various functions attributed to the IMD 16 and the processor 80 herein. The memory 82 may include any volatile, non-volatile, magnetic, optical, or electrical media, such as a random-access memory (RAM), read-only memory (ROM), non-volatile RAM (NVRAM), electrically erasable programmable ROM (EEPROM), flash memory, or any other digital media.

The processor 80 may include any one or more of a microprocessor, a controller, a digital signal processor (DSP), an application specific integrated circuit (ASIC), a field-programmable gate array (FPGA), or equivalent discrete or integrated logic circuitry. In some examples, processor 80 may include multiple components, such as any combination of one or more microprocessors, one or more controllers, one or more DSPs, one or more ASICs, or one or more FPGAs, as well as other discrete or integrated logic circuitry. The functions attributed to processor 80 herein may be embodied as software, firmware, hardware, or any combination thereof. The processor 80 controls the stimulation generator 84 to select a therapy mode (e.g., select one or more of an inhibited pacing mode, ventricular fusion pacing mode, atrioventricular synchronous pacing mode, atrial fibrillation pacing mode, etc.) and deliver stimulation therapy to the heart 12 according to the selected pacing mode, which may be stored in the memory 82, and various sensing (e.g., atrial depolarizations or activations, ventricular atrial depolarizations or activations, heartrate, P-wave-to-R-wave intervals, etc.). Specifically, the processor 80 may control the stimulation generator 84 to deliver electrical pulses with amplitudes, pulse widths, frequency, or electrode polarities specified by the selected one or more therapy programs and therapy modes.

In some embodiments, the cardiac conduction system pacing lead 18 may be operably coupled to the electrode 75, which may be used to monitor or pace the RA. The stimulation generator 84 may be electrically coupled to the electrodes 40, 42, 94, 48, 50, 58, 75, 62, 64, and 66, e.g., via conductors of the respective lead 23, 20, 18 or, in the case of housing electrode 58, via an electrical conductor disposed within the housing 60 of the IMD 16. The stimulation generator 84 may be configured to generate and deliver electrical stimulation therapy to the heart 12. For example, the stimulation generator 84 may deliver defibrillation shocks to the heart 12 via at least two of the electrodes 58, 62, 64, 66. The stimulation generator 84 may deliver pacing pulses via the ring electrodes 40, 94, 48 coupled to the leads 23, 20, 18, respectively, and/or the helical electrodes 42, 94, 50 of the leads 23, 20, or 18, respectively. In various embodiments, the cardiac conduction system pacing therapy can be delivered through the cardiac conduction system pacing lead 18 that is connected to an atrial, right ventricular, or left ventricular connection port of the connector block 34. In some examples, the stimulation generator 84 delivers pacing, cardioversion, or defibrillation stimulation in the form of electrical pulses. In other examples, the stimulation generator 84 may deliver one or more of these types of stimulation in the form of other signals, such as sine waves, square waves, or other substantially continuous time signals.

The stimulation generator 84 may include a switch module and the processor 80 may use the switch module to select, e.g., via a data/address bus, which of the available electrodes are used to deliver defibrillation shocks or pacing pulses. The switch module may include a switch array, switch matrix, multiplexer, or any other type of switching device suitable to selectively couple stimulation energy to selected electrodes.

The sensing module 86 monitors signals from at least one of the electrodes 40, 42, 94, 48, 50, 58, 75, 62, 64 or 66 in order to monitor electrical activity of the heart 12, e.g., via electrical signals, such as electrocardiogram (ECG) signals and/or electrograms (EGMs). The sensing module 86 may also include a switch module to select which of the available electrodes are used to sense the heart activity. In some examples, the processor 80 may select the electrodes that function as sense electrodes via the switch module within the sensing module 86, e.g., by providing signals via a data/address bus. In some examples, the sensing module 86 includes one or more sensing channels, each of which may include an amplifier. In response to the signals from the processor 80, the switch module may couple the outputs from the selected electrodes to one of the sensing channels.

In some examples, one channel of the sensing module 86 may include an R-wave amplifier that receives signals from the electrodes 94, which are used for pacing and sensing proximate to the LV of the heart 12. Another channel may include another R-wave amplifier that receives signals from the electrodes 48, 50, which are used for pacing and sensing in the RV of the heart 12. In some examples, the R-wave amplifiers may take the form of an automatic gain-controlled amplifier that provides an adjustable sensing threshold as a function of the measured R-wave amplitude of the heart rhythm.

In addition, in some examples, one channel of the sensing module 86 may include a P-wave amplifier that receives signals from electrodes the 40, 42, which are used for pacing and sensing in the RA of heart 12. In some examples, the P-wave amplifier may take the form of an automatic gain-controlled amplifier that provides an adjustable sensing threshold as a function of the measured P-wave amplitude of the heart rhythm. Examples of R-wave and P-wave amplifiers are described in U.S. Pat. No. 5,117,824 to Keimel et al., which issued on Jun. 2, 1992, and is entitled, “APPARATUS FOR MONITORING ELECTRICAL PHYSIOLOGIC SIGNALS,” and is incorporated herein by reference in its entirety. Other amplifiers may also be used. Furthermore, in some examples, one or more of the sensing channels of the sensing module 86 may be selectively coupled to the housing electrode 58, or the elongated electrodes 62, 64, or 66, with or instead of one or more of the electrodes 40, 42, 94, 48 or 50, e.g., for unipolar sensing of R-waves or P-waves in any of the chambers 26, 28, or 32 of the heart 12.

In some examples, the sensing module 86 includes a channel that includes an amplifier with a relatively wider pass band than the R-wave or P-wave amplifiers or a high-resolution amplifier with relatively narrow-pass band for His bundle or bundle branch potential recording. Signals from the selected sensing electrodes that are selected for coupling to this wide-band amplifier may be provided to a multiplexer, and thereafter converted to multi-bit digital signals by an analog-to-digital converter for storage in the memory 82 as an electrogram (EGM). In some examples, the storage of such EGMs in the memory 82 may be under the control of a direct memory access circuit. The processor 80 may employ digital signal analysis techniques to characterize the digitized signals stored in memory 82 to detect and classify the patient's heart rhythm from the electrical signals. The processor 80 may detect and classify the heart rhythm of the patient by employing any of the numerous signal processing methodologies known in the art.

If the IMD 16 is configured to generate and deliver pacing pulses to the heart 12, the processor 80 may include pacer timing and control module, which may be embodied as hardware, firmware, software, or any combination thereof. The pacer timing and control module may include a dedicated hardware circuit, such as an ASIC, separate from other the processor 80 components, such as a microprocessor, or a software module executed by a component of the processor 80, which may be a microprocessor or ASIC. The pacer timing and control module may include programmable counters which control the basic time intervals associated with DDD, VVI, DVI, VDD, AAI, DDI, DDDR, VVIR, DVIR, VDDR, AAIR, DDIR and other modes of single and dual chamber pacing. In the aforementioned pacing modes, “D” may indicate dual chamber, “V” may indicate a ventricle, “I” may indicate inhibited pacing (e.g., no pacing), and “A” may indicate an atrium. The first letter in the pacing mode may indicate the chamber that is paced, the second letter may indicate the chamber in which an electrical signal is sensed, and the third letter may indicate the chamber in which the response to sensing is provided.

Intervals defined by the pacer timing and control module may include atrial and ventricular pacing escape intervals, refractory periods during which sensed P-waves and R-waves are ineffective to restart timing of the escape intervals, and the pulse widths of the pacing pulses. As another example, the pace timing and control module may define a blanking time period and provide signals from sensing module 86 to blank one or more channels, e.g., amplifiers, for a period during and after delivery of electrical stimulation to the heart 12. The durations of these intervals may be determined by the processor 80 in response to stored data in the memory 82. The pacer timing and control module may also determine the amplitude of the cardiac pacing pulses.

During pacing, escape interval counters within the pacer timing/control module may be reset upon sensing of R-waves and P-waves. The stimulation generator 84 may include pacer output circuits that are coupled, e.g., selectively by a switching module, to any combination of the electrodes 40, 42, 94, 48, 50, 58, 75, 62, or 66 appropriate for delivery of a bipolar or unipolar pacing pulse to one of the chambers of the heart 12. The processor 80 may reset the escape interval counters upon the generation of pacing pulses by stimulation generator 84, and thereby control the basic timing of cardiac pacing functions, including anti-tachyarrhythmia pacing.

In some examples, the processor 80 may operate as an interrupt driven device and is responsive to interrupts from pacer timing and control module, where the interrupts may correspond to the occurrences of sensed P-waves and R-waves and the generation of cardiac pacing pulses. Any necessary mathematical calculations to be performed by the processor 80 and any updating of the values or intervals controlled by the pacer timing and control module of the processor 80 may take place following such interrupts. A portion of the memory 82 may be configured as a plurality of recirculating buffers, capable of holding series of measured intervals, which may be analyzed by the processor 80 in response to the occurrence of a pace or sense interrupt to determine whether the patient's heart 12 is presently exhibiting atrial or ventricular tachyarrhythmia.

The telemetry module 88 includes any suitable hardware, firmware, software, or any combination thereof for communicating with another device, such as the programmer 24. Under the control of the processor 80, the telemetry module 88 may receive downlink telemetry from and send uplink telemetry to the programmer 24 with the aid of an antenna, which may be internal and/or external. The processor 80 may provide the data to be uplinked to the programmer 24 and the control signals for the telemetry circuit within the telemetry module 88, e.g., via an address/data bus. In some examples, the telemetry module 88 may provide received data to the processor 80 via a multiplexer.

The various components of the IMD 16 are coupled to the power source 90, which may include a rechargeable or non-rechargeable battery. A non-rechargeable battery may be selected to last for several years, while a rechargeable battery may be inductively charged from an external device, e.g., on a daily or weekly basis.

The illustrative systems, devices, and methods described herein may provide adaptive cardiac conduction system pacing therapy that may select an appropriate cardiac conduction system pacing therapy mode based on one or more conditions or parameters measured from the patient.

The illustrative devices and methods described herein may provide and use monitored electrical activity (e.g., EGM, etc.) to determine various cardiac physiological conditions to provide effective pacing modes for use in cardiac therapy such as, for example, cardiac resynchronization therapy (CRT), biventricular pacing, LBB pacing (with or without atrial pacing), LV septal pacing, LBB block mode pacing; RBB pacing, RV septal pacing, RBB block mode pacing, etc. Further, the monitored electrical activity may be used to determine a difference in monitored electrical activity indicative of LV and RV depolarization, which may in turn be used to determine various cardiac physiological conditions. As will be described further herein, electrical activity of the heart may be monitored for specific areas of the heart, such as a specific chamber (e.g., RV, LV), and may be acquired using various devices as described herein (e.g., EGM).

If the determined time difference from at least one of (Ap) and (As) to LV depolarization is above an AV conduction threshold and the determined time difference from at least one of (Ap) and (As) to RV depolarization is above the AV conduction threshold, this may indicate AV block. If no AV block is determined, then the device, system, and method may continue to determine other cardiac physiological conditions. If AV block is determined, then the device, system, and method may or may not continue to determine other cardiac physiological conditions as discussed herein.

The difference (in monitored electrical activity indicative of LV and RV depolarization) may be determined, for example, by analyzing the monitored electrical activity. In one embodiment, the monitored electrical activity may include one or more EGM signals as monitored from the RBB and the LBB as described herein. The difference may be determined, or measured, as the time difference between the fiducial point of the monitored electrical activity using an electrode such as a LBB electrode 50 indicative of LV depolarization and the fiducial point of the monitored electrical activity using an electrode such as a RBB electrode 48 indicative of RV depolarization. For example, the monitored electrical activity indicative of LV depolarization may be determined, or measured, as the time point when the EGM signal as measured using the LBB electrode crosses the isoelectric line prior to the major QRS waveform or morphology. The monitored electrical activity indicative of RV depolarization may be determined, or measured, as the time point when the EGM signal as measured using the RBB electrode crosses the isoelectric line prior to the major QRS waveform or morphology. The isoelectric line denotes resting membrane potentials and has no positive or negative charges of electricity to create deflections. It is to be understood that any other EGM signal parameter may be used. The determined fiducial points, in any case, may be used to determine the difference between the fiducial points and thus determine various cardiac physiological conditions.

For example, the EGM signal parameter, and the resulting determined time difference between the parameter of the left and right ventricles, may be monitored to determine LBB block, RBB block, and normal AV conduction. If the determined time difference from the parameter of the LV to the parameter of the RV is negative, this may indicate LBB block. If the determined time difference from the parameter of the LV to the parameter of the RV is positive and above an RBB threshold, this may indicate RBB block. If the determined time difference from the parameter of the LV to the parameter of the RV is positive and below a normal conduction threshold, this may indicate normal AV conduction (e.g., no LBB or RBB block).

The IMD may utilize the determined cardiac physiological conditions, for example, to select and deliver a pacing configuration (e.g., cardiac conduction system pacing, myocardial pacing, or other pacing modes). Additionally, the IMD may adjust one or more pacing parameters within the selected pacing configuration to optimize the selected pacing configuration (e.g., pacing pulse voltage, pacing rate, etc.). Additionally, the determined difference between the parameter of the left and right ventricles may be monitored over time to ensure that continuing cardiac system pacing is optimized for the patient. Illustrative pacing therapy may be delivered using the devices as described in FIGS. 2-4.

Additionally, many cardiac conduction system pacing therapy systems may not include electrodes positioned proximate the atria (e.g., the right atrium) so as to acquire and monitored near-field, or local, atrial electrical activity. For instance, cardiac conduction system pacing therapy systems may only include electrodes positioned outside of the right atrium. In at least one embodiment, cardiac conduction system pacing therapy systems may only include single chamber devices having electrodes implanted in the RV. As a result, such systems may utilize far-field electrical activity using one or more electrodes positioned outside of the right atrium of the patient's heart. Such far-field electrical activity may be processed to determine the P-waves, which are indicative of atrial depolarization, so as to be used to time and deliver cardiac conduction system pacing therapy.

For example, the single chamber device may include a single lead extending through the right atrium into the RV as shown in FIGS. 3A-3B. The dual chamber device may include one or more leads or leadless devices. For example, the dual chamber device may include a first lead extending into the right atrium and a second lead extending into the RV as shown in FIG. 2B. The three-chamber device may include multiple leads or leadless devices. For example, the three-chamber device may include a first lead extending into the RV, a second lead extending into the coronary sinus to a region adjacent to the free wall of the LV, and a third lead extending into the right atrium, as shown in FIG. 2A.

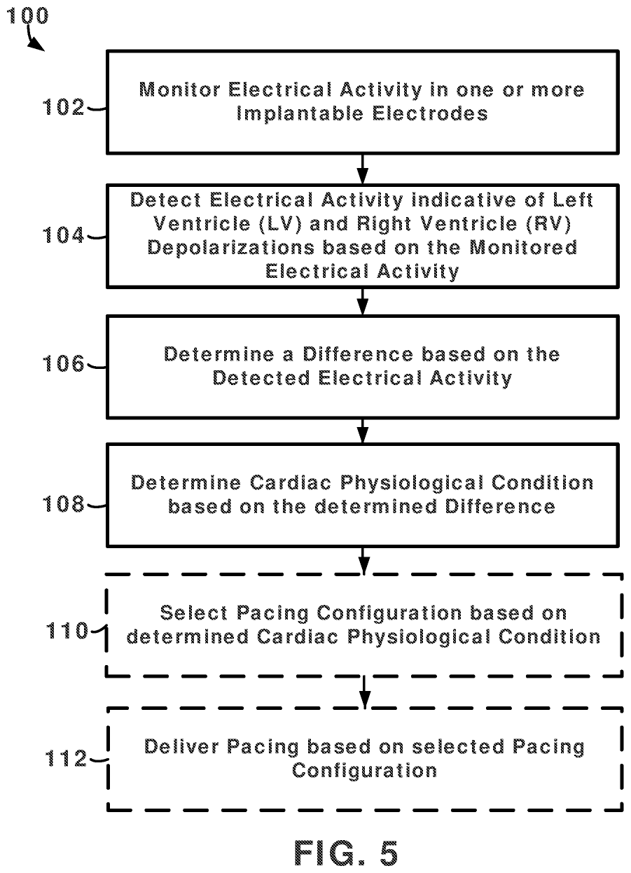

An illustrative method 100 of determining a cardiac physiological condition based on a determined difference, where the determined difference is based on monitored electrical activity (and which method 100 may be utilized by the devices of FIGS. 2-3) is depicted in FIG. 5. The method 100 may include ongoing delivery of cardiac system pacing therapy (not shown) as a preset for a patient. During the delivery of the therapy, cardiac electrical activity may be monitored using one or more of the electrodes as part of the pacing regimen. In particular, one or more of intrinsic atrial depolarizations or activations, paced atrial depolarizations or activations, intrinsic ventricular depolarizations or activations, and paced ventricular depolarizations or activations may be sensed in the monitored electrical activity so as to be used to detect various cardiac physiological conditions. Periodically, in one embodiment, the cardiac system pacing therapy being delivered to the ventricle(s) may be paused. In other words, ventricle pacing therapy may be temporarily suspended or inhibited such that intrinsic cardiac electrical activity may be monitored using one or more of the electrodes, and the monitored electrical activity may be used to detect various cardiac physiological conditions.

In at least one embodiment, the ongoing delivery of cardiac system pacing therapy may include a set atrioventricular delay. During the paused period, the atrioventricular delay may be set, or configured to, a time value that is long enough to observe intrinsic conduction to the ventricles for intrinsic depolarization or activation while still providing pacing therapy if required. In other words, the atrioventricular delay may be extended or lengthened to allow for intrinsic ventricular activation but still deliver ventricular pacing if no intrinsic ventricular activation occurs (e.g., due to AV block, etc.). Determination of various cardiac physiological conditions is described herein with reference to FIGS. 5-15.

The method 100 may include pausing any ongoing pacing or setting a long enough atrioventricular delay to allow for intrinsic conduction, as described herein, and the method 100 may include monitoring electrical activity in one or more implantable electrodes 102.

Monitoring electrical activity in the heart may include monitoring near-field electrical signals using the systems and devices as described above. Monitoring electrical activity may be accomplished using the sensing module 86, for example, as described herein. Electrical activity may include a near-field electrogram (EGM) signal as sensed from the at least one implanted electrode. In one or more embodiments, electrical activity may be monitored in the RV and LV of the heart. In alternate embodiments, electrical activity may be monitored in, on, or around the LBB, RBB, or left or right ventricular septum, etc. The sensing module 86 or other sensing apparatus may monitor the electrical activity 102 (e.g., EGM signal) during intrinsic heartbeats.

In one embodiment, monitoring electrical activity 102 may include use of one or more implanted electrodes as discussed herein with respect to FIGS. 1-4, such as a ring electrode to a housing electrode unipolar EGM, a tip electrode to a housing electrode unipolar EGM, an RV coil electrode to a housing electrode unipolar EGM, and an atrial ring electrode to a housing electrode unipolar EGM. A tip electrode may be an electrode located on the tip of any lead as described above. A coil electrode may be an electrode shaped in or on a coil and located proximal to the tip of the lead along the lead body. A ring electrode may be an electrode shaped in a ring around the lead body and located proximal to the tip of the lead along the lead body. A housing electrode may be an electrode located in or about the housing of the IMD. Location identifiers such as RBB are examples of possible electrode locations. For example, the “atrial” location may be one of either atrium. In alternative embodiments, bipolar EGM signals may be monitored.

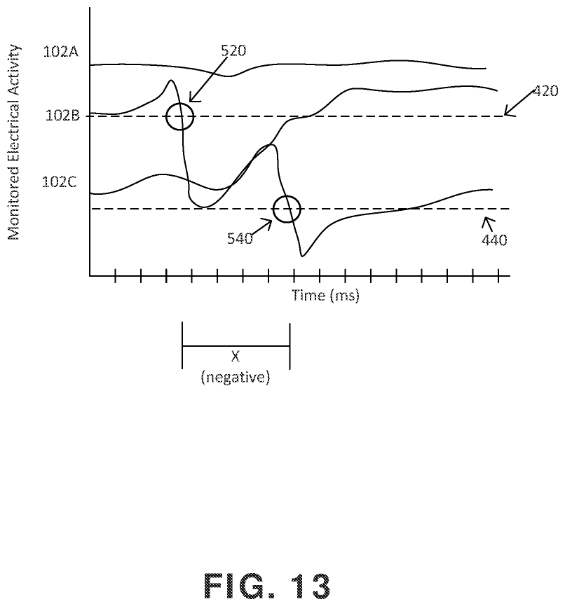

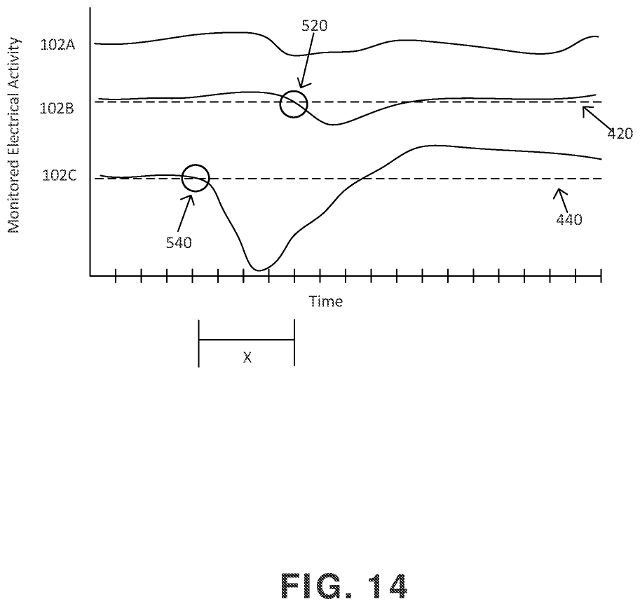

In one embodiment, and as described above, an RBB unipolar electrode (e.g., RBB ring to can unipolar implanted electrogram using electrode 48) may be used to monitor electrical activity of the right ventricle along the RBB. The RBB unipolar electrode may be used to produce an EGM signal 102B, as illustrated and labeled in FIGS. 13-15, illustrating at least part of the monitored electrical activity of the method 100 of FIG. 5. An LBB unipolar electrode (e.g., LBB tip to can unipolar implanted electrogram using electrode 50) may be used to monitor electrical activity of the left ventricle along the LBB. The LBB unipolar electrode may be used to produce an EGM signal 102C, as illustrated and labeled in FIGS. 13-15, illustrating at least part of the monitored electrical activity of the method 100 of FIG. 5. Additionally, FIGS. 13-15 illustrates an EGM signal 102A, which may be produced using a right atrial ring to housing electrode unipolar implanted electrogram (similar to right atrial electrode 75 discussed herein). Still further, the device or system may also include an LV implantable electrode that is implanted in the left ventricle, but not in the cardiac conduction system, such as described herein with respect to lead 20. The LV electrode 94 may be used additionally or alternatively to monitor electrical activity of the method 100 of FIG. 5.

In one embodiment, each of the EGM signals 102B and 102C may be any EGM signal obtained from the RBB and LBB electrodes as discussed herein. In another embodiment, each of the EGM signals 102B and 102C may be a single EGM signal that has passed through a low pass filter, or may be, for examples, an averaged EGM signal obtained from more than one electrode or obtained over more than one heartbeat. The isoelectric line for RBB electrode EGM 102B is designated with line 420, and the isoelectric line for LBB electrode EGM 102C is designated with line 440.

The monitored electrical activity can be based on, for example, the patient's intrinsic ventricle cardiac electrical activity during a pause in ventricle pacing described herein following an RA sensed or paced cardiac electrical activity.

The method 100 may further include detecting electrical activity indicative of LV and RV depolarizations 104 based on the monitored electrical activity 102 (and as illustrated in FIGS. 5-15 and described further herein). The detected electrical activity indicative of LV depolarization can be based on the monitored electrical activity from the LBB electrode, for example. The detected electrical activity indicative of RV depolarization can be based on the monitored electrical activity from the RBB electrode. The detected electrical activity indicative of LV depolarization and indicative of RV depolarization can be determined based on the monitored electrical activity from an initial monitored heartbeat, based on a second monitored heartbeat, based on a subsequent monitored heartbeat, based on an average heartbeat, etc.

In one embodiment, the RV depolarization is illustrated using the EGM signal 102B and the LV depolarization is illustrated using the EGM signal 102C. For example, the RV depolarization is detected based on an RBB fiducial point 520 in the monitored EGM signal 102B, and LV depolarization is detected based on an LBB fiducial point 540 in the monitored EGM signal 102C. Fiducial point 520 is illustrating the monitored QRS deflection indicative of RV depolarization via the RBB electrode. Fiducial point 540 is illustrating the monitored QRS deflection indicative of LV depolarization via the LBB electrode.

In another embodiment, LV depolarization is detected based on an LV fiducial point (not shown) in the monitored EGM signal of a septal LV electrode (as discussed herein with respect to FIG. 2A). Hereinafter, the LBB fiducial point is understood to encompass both the EGM signal using the LBB electrode or an EGM signal using the LV electrode.

The fiducial point discussed herein may be any measurable point within the near-field electrical activity signal or derivatives thereof (e.g., the differential signal) that corresponds, or correlates, to the actual respective chamber depolarization. To determine the fiducial point, the devices and systems as described herein may identify the fiducial point using any calculations or methodologies, such as by using sensing module 86 as described herein, processor 80 as described herein, etc. Some examples of fiducial points may include one or more of the following: onset of a QRS morphology or any specific points therein, maximum signal amplitude, minimum signal amplitude, minimum and maximum signal slopes, the point where the monitored electrical activity crosses the isoelectric line 420, 440 (which may be located, for example, at 0 Volts), any other monitored electrical activity signal waveform, etc. In one embodiment, the fiducial point 520, 540 is where the monitored electrical activity crosses the isoelectric line 420, 440, as discussed herein.

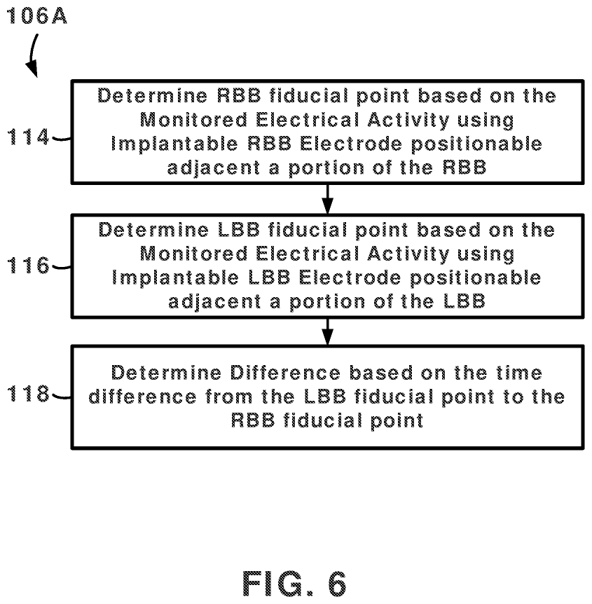

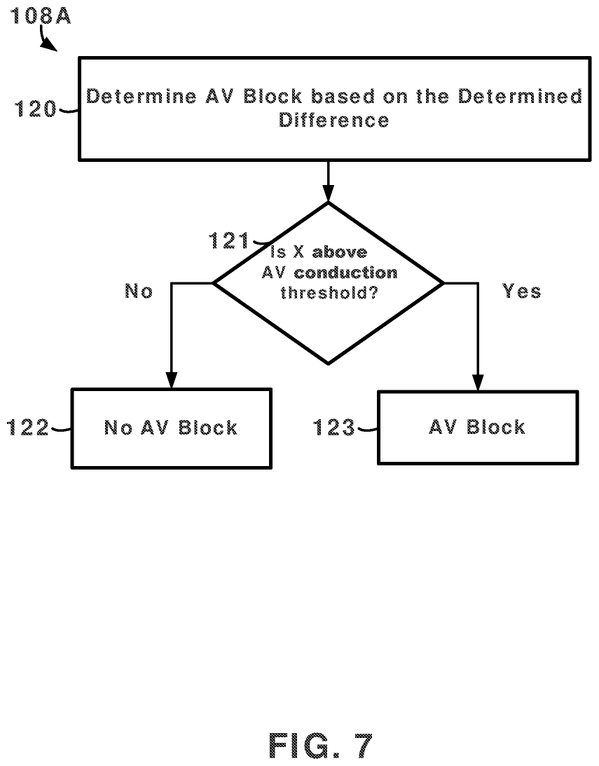

The method 100 may further include determining a difference (illustrated as “X” in FIGS. 13-15) based on the detected electrical activity 106. In one embodiment, determining the difference X may include measuring any detectable time difference from the LBB fiducial point 540 to the RBB fiducial point 520 (optional method 106A and box 118 as illustrated in FIG. 6). The difference X may be positive if the LBB fiducial point 540 occurs prior to the RBB fiducial point 520, and the difference X may be negative if the RBB fiducial point 520 occurs before the LBB fiducial point 540. In alternative embodiments, for example, the difference X may include measuring any detectable time difference from the RBB fiducial point 520 to the LBB fiducial point 540 such that X is positive if the RBB fiducial point occurs prior to the LBB fiducial point and X is negative if the LBB fiducial point occurs prior to the RBB fiducial point. In further alternative embodiments, the difference X may include measuring any detectable time difference from the LV depolarization to the RV depolarization, or vice versa. For purposes of this disclosure, the difference X is discussed as any detectable time difference from the LBB fiducial point 540 to the RBB fiducial point 520 for determining LBB block, RBB block, and intrinsic AV conduction (optional method 106A and box 118 as illustrated in FIG. 6). For purposes of this disclosure, the difference X is also discussed as any detectable time difference from at least one of (Ap) or (As) to LV depolarization and the time difference from at least one of (Ap) or (As) to RV depolarization for determining AV block (optional method 108A and box 121 as illustrated in FIG. 7). Thus, the difference X is always based on the detected electrical activity 106.

In one embodiment, the EGM signal using the RBB electrode 102B may include the RBB fiducial point 520, which may be identified during an intrinsic heartbeat during the pause (or intrinsic conduction during the long atrioventricular delay) in pacing described herein. The monitored electrical activity from an intrinsic heartbeat may be monitored during a first heartbeat after pacing is paused (or the atrioventricular delay is lengthened), or it may be monitored during a second or subsequent or average heartbeat after pacing is paused (or the atrioventricular delay is lengthened).

In an alternative embodiment, the heart may be paced using an RA electrode 75 positionable adjacent a portion of the patient's RA. Such electrode may be as described above with respect to FIG. 3A. The computing apparatus is further configured to deliver RA pacing using the RA electrode 75. Thus, in any embodiment, the heart is either intrinsically beating or is paced via the RA. In such alternative embodiment, the atrioventricular delay may be set to be long enough to allow for intrinsic conduction. The monitored electrical activity from a paced heartbeat may be monitored during a first paced heartbeat, or it may be monitored during a second or subsequent or average paced heartbeat.

The RBB fiducial point 520 may be identified in various ways, as described herein. For example, the RBB fiducial point 520 may be determined based on the monitored electrical activity of the implantable RBB electrode 48 positionable adjacent a portion of the RBB 8b (as illustrated in FIG. 6, box 114). Similarly, the EGM signal using the LBB electrode 102C may include the LBB fiducial point 540, which may be identified in the same intrinsic heartbeat as the RBB fiducial point 520. For example, the LBB fiducial point 540 may be determined based on the monitored electrical activity of the implantable LBB electrode 50 positionable adjacent a portion of the LBB 8a (as illustrated in FIG. 6, box 116). In this described embodiment, the electrode 48 is a ring electrode, and the electrode 50 is a tip electrode, and both are coupled to the IMD housing, and any housing electrode located therein (as described herein).

In another embodiment, the one or more implantable electrodes further include a LV electrode positionable adjacent a portion of the patient's LV, such as described herein with respect to lead 20. Detecting electrical activity indicative of LV depolarization may be based on the monitored electrical activity using the LV electrode 94, as opposed to, or in addition to, the LBB electrode.

As discussed herein, the EGM signal and the determined difference X in time between fiducial points may be used to determine a cardiac physiological condition. Also as discussed herein, the EGM signal and the determined difference X may be monitored and measured periodically to ensure accuracy of the determined difference X, and the relative consistency of the determined difference X over time, or at one or more paced settings, may also be averaged to calculate the determined difference X. Signal noise can make the waveform cross the isoelectric line more than once, or more frequently, making it difficult to detect the fiducial points based on the isoelectric line. The noise may be filtered such that, for example, crossing the isoelectric line may mean crossing above a hysteresis threshold value added to the isoelectric line.

The relative consistency of the determined difference X (as defined anywhere in this specification) may be determined over time. For example, the relative consistency of the determined difference X may be defined as having the difference X at one intrinsic heartbeat within about 9% of the difference X at a prior intrinsic heartbeat (e.g., the heartbeat directly prior, or another prior heartbeat). In other embodiments, the consistency as defined may remain within about 5%, or about 10%, or about 15 %, or about 20%, or about 25 %, or about 30%, or about 35%, or about 40%, or about 50%, or about 60%, or about 70%, or about 80%, or about 90% of the difference X at a prior intrinsic heartbeat. In another embodiment, the consistency may be defined in terms of a ratio as opposed to a percentage or may be defined in terms of a set difference X time value, or threshold value. Still further, for example, the consistency may be any consistency within a selected range.

After determining the difference X based on any detectable time difference from at least one of (Ap) or (As) to LV depolarization and the time difference from at least one of (Ap) or (As) to RV depolarization, the method 100 may further include determining a cardiac physiological condition based on the determined difference X 108. As illustrated in FIG. 7, the method 108A may include determining AV block based on the determined difference 120. Such determination may analyze if the time (e.g., milliseconds) between at least one of (Ap) and (As) and the depolarization of the LV, and the time between at least one of (Ap) and (As) and the depolarization of the RV are above an AV conduction threshold 121. If the determined times are above the AV conduction threshold, this indicates AV block 123. If the determined times are not above the AV conduction threshold, this indicates no AV block 122.

The AV conduction threshold may be preset (such as by a physician or other medical professional) for the patient based on the patient's physiological condition and needs. The AV conduction threshold may be based on a statistical analysis of a certain population with shared characteristics. The AV conduction threshold may be determined from a template heartbeat. In another embodiment, AV block may be determined by monitoring, for example, the PR interval from the electrogram or electrograms of the implanted device, or the QRS morphology to determine if the patient has AV block.

The AV conduction threshold may be between about 200 ms and about 450 ms. In at least one embodiment, the AV conduction threshold is 300 ms. In other embodiments, the AV conduction threshold may be greater than or equal to 200 ms, greater than or equal to 250 ms, greater than or equal to 275 ms, greater than or equal to 300 ms, greater than or equal to 350 ms, greater than or equal to 400 ms, greater than or equal to 450 ms, etc. and/or less than or equal to 450 ms, 400 ms, 350 ms, 300 ms, 250 ms, 200 ms, etc.

In any embodiment, if no AV block is determined, then the device, system, and method may continue to determine other cardiac physiological conditions. If AV block is determined, then the device, system, and method may not continue to determine other cardiac physiological conditions as discussed herein. Additionally, if the LV depolarization and the RV depolarization occur after the atrial activity at or greater than an AV conduction threshold, then the patient may have AV conduction block, and the device, system, and method may not continue to determine other cardiac physiological conditions as discussed herein.

Still further, if there is ectopy in the heartbeat as monitored using the monitored electrical activity, there may be an inconsistent PP interval or an inconsistent RR interval between the monitored heartbeat and the preceding average heart rate. If there is ectopy, the device, system, and method may not continue to determine other cardiac physiological conditions as discussed herein. If there is not ectopy, the device, system, and method may continue to determine other cardiac physiological conditions as discussed herein.

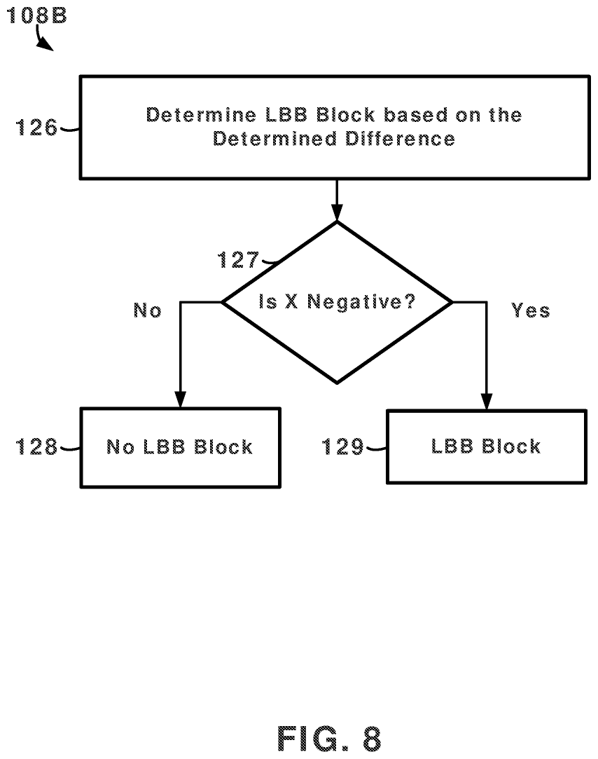

As illustrated in FIG. 8, the method 108B may include determining LBB block based on the determined difference 126. Such determination may analyze if the determined difference X in time (e.g., milliseconds) from the depolarization of the LV to the depolarization of the RV is negative 127. This is illustrated in FIG. 13, where the RBB fiducial point 520 occurs before the LBB fiducial point 540 and hence produces a negative time difference. If the determined time difference X is negative, this indicates LBB block 129. If the determined time difference X is not negative, this indicates no LBB block 128. Additionally, in some cases the determined difference may be negative (e.g., due to signal noise), but the patient is not experiencing LBB block. In alternative embodiments, if the determined difference X is negative and below an LBB threshold (not shown), this indicates LBB block.

The LBB threshold (not shown) may be preset (such as by a physician or other medical professional) for the patient and based on the patient's physiological condition and needs. The LBB threshold may be based on a statistical analysis of a certain population with shared characteristics. The LBB threshold may be determined from a template heartbeat. The LBB threshold may be between about 0 ms and about −40 ms. In at least one embodiment, the LBB threshold is −10 ms. In other embodiments, the LBB threshold may be greater than or equal to −40 ms, greater than or equal to −30 ms, greater than or equal to −20 ms, greater than or equal to −10 ms, greater than or equal to −5 ms, greater than or equal to 0 ms, etc. and/or less than or equal to 0 ms, −5 ms, −10 ms, −20 ms, −30 ms, −40 ms, etc.

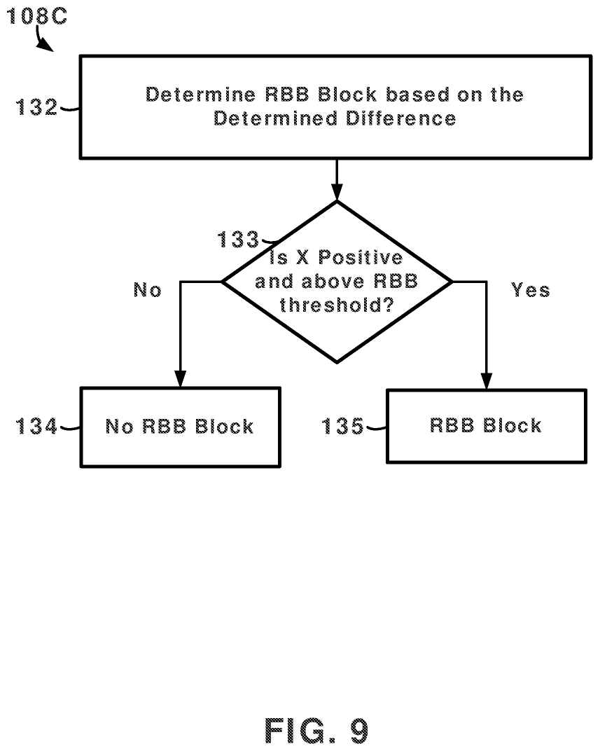

As illustrated in FIG. 9, the method 108C may include determining RBB block based on the determined difference 132. Such determination may analyze if the determined difference X in time (e.g., milliseconds) from the depolarization of the LV to the depolarization of the RV is positive and above an RBB threshold 133. If the determined time difference X is positive and above the RBB threshold, this indicates RBB block 135. If the determined time difference X is not positive or is not above the RBB threshold, this indicates no RBB block 134. This is also illustrated in FIG. 14, where the RBB fiducial point 520 occurs after the LBB fiducial point 540 (hence a positive time difference), and the value of the determined difference X is above the RBB threshold.

The RBB threshold may be the same or different than the AV conduction threshold. The RBB threshold may be preset (such as by a physician or other medical professional) for the patient and based on the patient's physiological condition and needs. The RBB threshold may be based on a statistical analysis of a certain population with shared characteristics. The RBB threshold may be determined from a template heartbeat. The RBB threshold may be between about 25 milliseconds (ms) and about 40 ms. In at least one embodiment, the RBB threshold is 30 ms. In other embodiments, the RBB threshold may be greater than or equal to 30 ms, greater than or equal to 32 ms, greater than or equal to 34 ms, greater than or equal to 36 ms, greater than or equal to 38 ms, greater than or equal to 40 ms, etc. and/or less than or equal to 40 ms, 38 ms, 36 ms, 34 ms, 32 ms, 30 ms, etc.

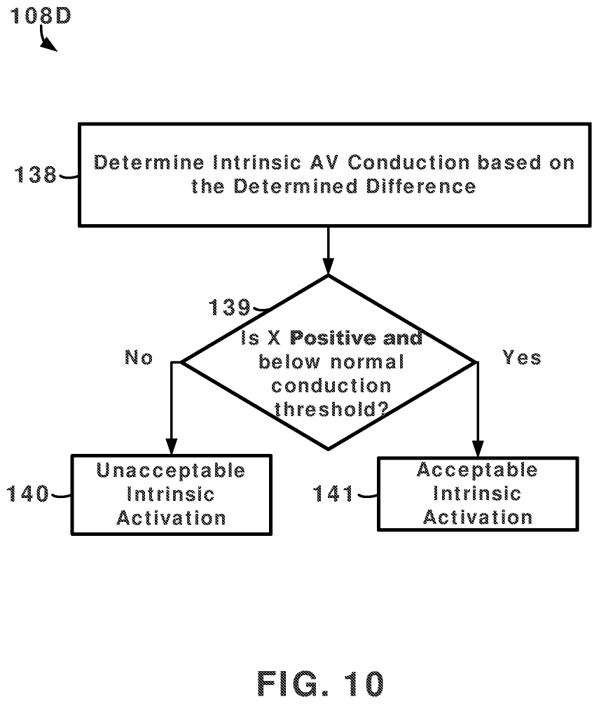

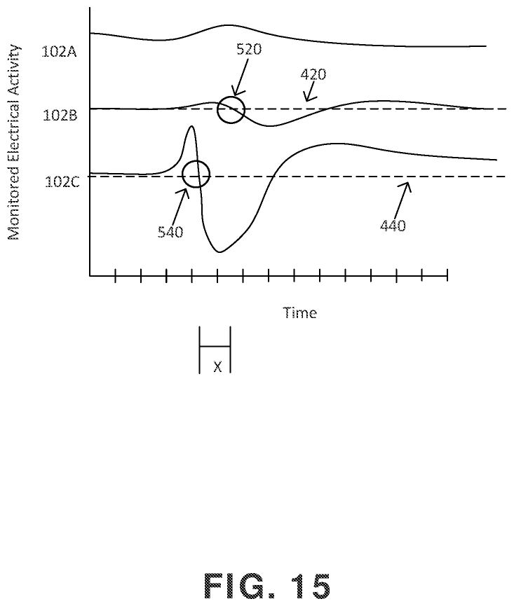

As illustrated in FIG. 10, the method 108D may include determining normal intrinsic AV conduction based on the determined difference 138. Such determination may analyze if the determined difference X in time (e.g., milliseconds) from the depolarization of the LV to the depolarization of the RV is positive (e.g., the left ventricle activates before the right ventricle) and below a normal conduction threshold 139. If the determined time difference X is positive and below the normal conduction threshold, this is acceptable intrinsic pacing 141. If the determined time difference X is not positive or is not below the normal conduction threshold, this indicates unacceptable intrinsic pacing 140. This is also illustrated in FIG. 15, where the RBB fiducial point 520 occurs after the LBB fiducial point 540, and the value of the determined difference X is below the normal conduction threshold. Acceptable intrinsic pacing is defined as intrinsic, non-paced heartbeats with normal AV conduction through the heart. Thus, no cardiac conduction system pacing is needed, and the heart's cardiac conduction system is functioning in a normal and healthy manner.

The normal conduction threshold may be the same or different than the AV conduction threshold and the RBB threshold. The normal conduction threshold may be preset (such as by a physician or other medical professional) for the patient and based on the patient's physiological condition and needs. The normal conduction threshold may be based on a statistical analysis of a certain population with shared characteristics. The normal conduction threshold may be determined from a template heartbeat. The normal conduction threshold may be between about 10 ms and about 40 ms. In at least one embodiment, the normal conduction threshold is 10 ms. In other embodiments, the normal conduction threshold may be greater than or equal to 15 ms, greater than or equal to 20 ms, greater than or equal to 25 ms, greater than or equal to 27 ms, greater than or equal to 30 ms, greater than or equal to 35 ms, greater than or equal to 40 ms, etc. and/or less than or equal to 40 ms, 35 ms, 30 ms, 25 ms, 20 ms, 15 ms, 10 ms, etc.

In alternative embodiments, acceptable intrinsic pacing may include a slightly negative determined difference X (e.g., due to signal noise). In such cases, the LBB threshold (not shown) is also used, because in some cases a slightly negative X may indicate acceptable intrinsic pacing and may not indicate LBB block. For example, the LBB threshold may be more negative than the normal conduction threshold. In such alternative embodiments, the normal conduction threshold may be between about −10 ms and about 40 ms. In at least one embodiment, the normal conduction threshold is 0 ms. In other embodiments, the normal conduction threshold may be greater than or equal to −10 ms, greater than or equal to 0 ms, greater than or equal to 10 ms, greater than or equal to 20 ms, greater than or equal to 30 ms, greater than or equal to 35 ms, greater than or equal to 40 ms, etc. and/or less than or equal to 40 ms, 35 ms, 30 ms, 25 ms, 20 ms, 10 ms, 0 ms, −10 ms, etc.

Additionally, if there is unblocked intrinsic AV conduction, the RBB fiducial point 520 and the LBB fiducial point 540 are generally equal in time. For example, the fiducial points may be spaced apart from one another by between about 0 ms and about 15 ms. In at least one embodiment, the difference between them is 0 ms. In other embodiments, the difference between them may be greater than or equal to 1 ms, greater than or equal to 2 ms, greater than or equal to 3 ms, greater than or equal to 4 ms, greater than or equal to 5 ms, greater than or equal to 6 ms, greater than or equal to 7 ms, greater than or equal to 10 ms,, greater than or equal to 15 ms, etc. and/or less than or equal to 15 ms, 10 ms, 7 ms, 6 ms, 5 ms, 4 ms, 3 ms, 2 ms, 1 ms, 0 ms, etc.

Such fiducial points (when there is unblocked intrinsic conduction) should be below the AV conduction threshold. If the AV delay is short, and there is a slight difference in the fiducial points (as described above), intrinsic activation may be allowed to continue. If the AV delay is not short, and there is a difference in the fiducial points, intrinsic activation may not be allowed to continue, and a pacing configuration may be selected.

A short AV delay may be between about 30 ms and about 70 ms. In at least one embodiment, the short AV delay is 50 ms. In other embodiments, the short AV delay may be greater than or equal to 30 ms, greater than or equal to 40 ms, greater than or equal to 50 ms, greater than or equal to 60 ms, greater than or equal to 70 ms, etc. and/or less than or equal to 70 ms, 60 ms, 50 ms, 40 ms, 30 ms, etc.

The time from (As) to the fiducial points as described above may be between about 100 ms and about 280 ms. In at least one embodiment, the time from (As) to the fiducial points is 200 ms. In other embodiments, the time from (As) to the fiducial points may be greater than or equal to 150 ms, greater than or equal to 175 ms, greater than or equal to 200 ms, greater than or equal to 225 ms, greater than or equal to 250 ms, etc. and/or less than or equal to 250 ms, 225 ms, 200 ms, 175 ms, 150 ms, etc.

The time from (Ap) to the fiducial points as described above may be between about 120 ms and about 300 ms. In at least one embodiment, the time from (Ap) to the fiducial points is 250 ms. In other embodiments, the time from (Ap) to the fiducial points may be greater than or equal to 120 ms, greater than or equal to 200 ms, greater than or equal to 250 ms, greater than or equal to 275 ms, greater than or equal to 300 ms, etc. and/or less than or equal to 300 ms, 275 ms, 250 ms, 225 ms, 120 ms, etc.

The method 100 of FIG. 5 may further optionally include selecting a pacing configuration (e.g., cardiac conduction system pacing, myocardial pacing, dual pacing) based on the determined cardiac physiological condition 110, such option as illustrated using an optional dashed-line box. The selected pacing configuration may be selected by a physician or other medical professional or may be selected by the device or system. If the selected pacing configuration including pacing with either or both the RBB ring electrode 48 and the LBB tip electrode 50, then pacing settings (e.g., voltage, pacing rate, etc.) may be optimized to save energy and provide the best possible treatment to the patient. If only one of the above electrodes is used in pacing, more energy saving is possible.

Various embodiments describing delivery of cardiac system pacing are discussed herein (e.g., using an IMD). Additionally, the method 100 may be performed more than once, so that a medical provider or user may periodically recheck the determined difference X, change pacing configurations, pacing configuration settings, etc., based on the determined difference X. Disease progression can make LBB or RBB block more permanent, and continued monitoring can ensure optimized treatment for the patient over time.

The method 100 may further optionally include delivering pacing based on the selected pacing configuration 112, and such delivery is also illustrated using an optional dashed-line box. Such pacing may be done using the devices as described with respect to FIGS. 2-4.

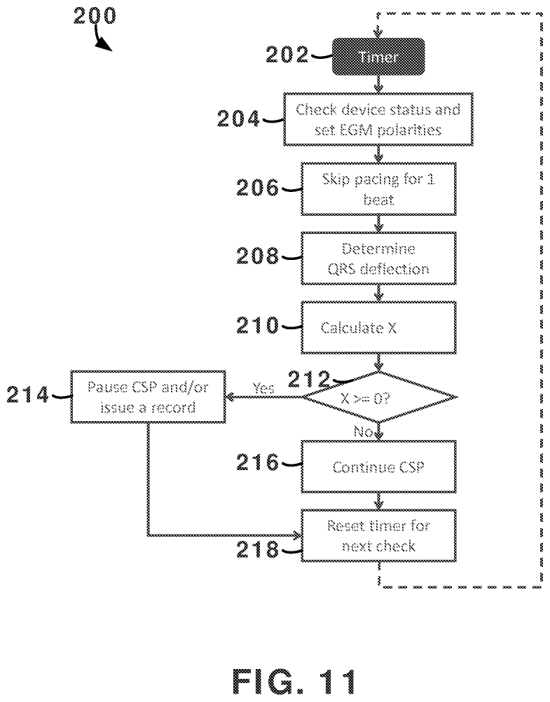

FIG. 11 is a block diagram of another illustrative method 200 of determining the difference X between QRS deflection of the left and right ventricles that may be utilized by the devices of FIGS. 2-4 and using one or more processes that are the same or similar to the methods illustrated in FIGS. 5-10. The method 200 of FIG. 11 may be applied to or integrated with a capture management feature as described herein, and further may be used to chronically track the trend of the determined difference X as a monitoring feature over time.

The method 200 includes setting a timer 202 to know when to pause any ongoing pacing to the patient. The method 200 further includes checking the IMD device status and setting EGM polarities of the one or more implanted leads and implanted electrodes of the device 204. The method 200 further includes skipping pacing for one heartbeat 206. This ensures that a fully intrinsic heartbeat is monitored, as opposed to a paced heartbeat. In alternative embodiments, the method may be include monitoring the fully intrinsic heartbeat to ensure, for example, that it is not ectopic, or that it is normal. Such a normal intrinsic heartbeat may be based on a statistical analysis of a certain population, or may be determined from a template normal heartbeat, or may be determined by monitoring QRS morphology such as QRS width, R-wave amplitude, RR interval, PR interval, etc., or any combination thereof. In further alternative embodiments, the heartbeat may not be skipped and 206 is not performed.

The method 200 further includes determining QRS deflection 208 for both the left and right ventricles. This may be similar to determining the LBB and RBB fiducial points as discussed herein. The method 200 may further include calculating the difference X between the QRS deflections (e.g., delta). This may be similar to determining the difference X as discussed herein. The method 200 may further include analyzing if the difference X is greater than or equal to zero at 212. If the difference X is greater than or equal to zero, any cardiac system pacing (CSP) may be paused and/or a record may be issued 214. If the difference X is not greater than or equal to zero, CSP may continue 216. The method 200 may further include resetting the timer for the next check 218, and the method 200 may restart again at the end of the timer.

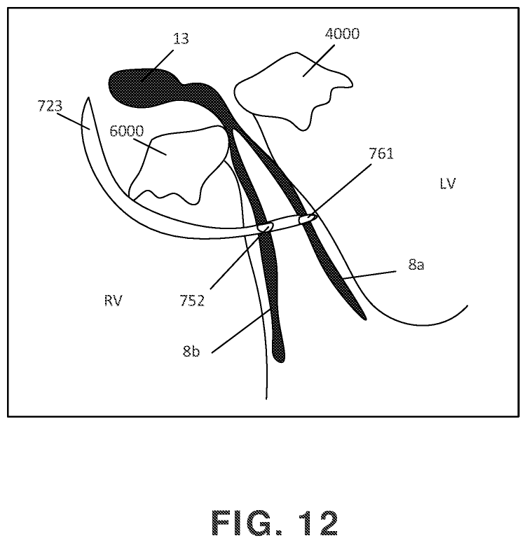

FIG. 12 is a simple illustrative diagram of a portion of a heart and a lead that may be utilized by the devices of FIGS. 2-4. The His bundle 13, the RBB 8b and the LBB 8a are all shaded or filled in. A tricuspid valve 6000 is located at the entrance to the RV, and a mitral valve 4000 is located at the entrance to the LV. The cardiac conduction system pacing therapy lead 23 is implanted as described herein, with the electrode 48 and the electrode 50 as described herein.

Various examples have been described. These and other examples are within the scope of the following claims. For example, a single chamber, dual chamber, or triple chamber pacemakers (e.g., CRT-P) or ICDs (e.g., CRT-D) devices can be used to implement the illustrative methods described herein.

### ILLUSTRATIVE EXAMPLES

While the present disclosure is not so limited, an appreciation of various aspects of the disclosure will be gained through a discussion of the specific illustrative examples provided below. Various modifications of the illustrative examples, as well as additional examples of the disclosure, will become apparent herein.

Example Ex1: An implantable medical device comprising:
a computing apparatus comprising processing circuitry and operably coupled to one or more implantable electrodes comprising a left bundle branch (LBB) electrode positionable adjacent a portion of a patient's LBB, and a right bundle branch (RBB) electrode positionable adjacent a portion of the patient's RBB, wherein the computing apparatus is configured to:
   monitor electrical activity using the LBB electrode and the RBB electrode;
   detect electrical activity based on the monitored electrical activity indicative of intrinsic left ventricle (LV) depolarization and indicative of intrinsic right ventricle (RV) depolarization;
   determine a temporal difference based on the detected electrical activity indicative of intrinsic LV depolarization and the detected electrical activity indicative of intrinsic RV depolarization; and
   determine whether there is a cardiac physiological condition based on the determined difference.
Example Ex2: A method comprising:
monitoring electrical activity using one or more implantable electrodes comprising a left bundle branch (LBB) electrode positioned adjacent a portion of a patient's LBB and using a right bundle branch (RBB) electrode positioned adjacent a portion of the patient's RBB;
detecting electrical activity based on the monitored electrical activity indicative of intrinsic left ventricle (LV) depolarization and indicative of intrinsic right ventricle (RV) depolarization;
determining a difference based on the detected electrical activity indicative of intrinsic LV depolarization and the detected electrical activity indicative of intrinsic RV depolarization; and
determining whether there is a cardiac physiological condition based on the determined difference.

Example Ex3: The implantable medical device as in Example Ex1 or the method as in Example Ex2, wherein detecting electrical activity indicative of intrinsic LV depolarization is based on the monitored electrical activity using the LBB electrode, and wherein detecting electrical activity indicative of intrinsic RV depolarization is based on the monitored electrical activity using the RBB electrode.

Example Ex4: The implantable medical device or the method as in any one of Examples Ex1-Ex3, wherein the one or more implantable electrodes further comprise a LV electrode positionable adjacent a portion of the patient's LV, and wherein detecting electrical activity indicative of intrinsic LV depolarization is based on the monitored electrical activity using the LV electrode.

Example Ex5: The implantable medical device or the method as in any one of Examples Ex1-Ex4, wherein the one or more implantable electrodes further comprise a right atrium (RA) electrode positionable adjacent a portion of the patient's RA, and wherein the computing apparatus is further configured to execute, or the method further comprises:
detecting intrinsic RA cardiac electrical activity or delivering RA pacing using the RA electrode.

Example Ex6: The implantable medical device or the method as in any one of Examples Ex1-Ex5, wherein the monitored electrical activity is based on at least one of the patient's intrinsic RA cardiac electrical activity and RA paced cardiac electrical activity, and wherein the detected electrical activity indicative of LV depolarization and indicative of RV depolarization is determined based on at least one of an initial monitored heartbeat or a second or subsequent monitored heartbeat.

Example Ex7: The implantable medical device or the method as in any one of Examples Ex1-Ex6, wherein the computing apparatus is further configured to execute, or the method further comprises:
selecting a pacing configuration based on the determined cardiac physiological condition; and
delivering pacing based on the selected pacing configuration using the one or more implanted electrodes.

Example Ex8: The implantable medical device or the method as in any one of Examples Ex1-Ex7, wherein the determined difference based on the detected electrical activity indicative of LV depolarization and the detected electrical activity indicative of RV depolarization is a time period extending from a fiducial point of the monitored electrical activity using the LBB electrode to a fiducial point of the monitored electrical activity using the RBB electrode.

Example Ex9: The implantable medical device or the method as in Example Ex8, wherein the fiducial point of the monitored electrical activity using the LBB electrode is an earliest monitored QRS deflection and wherein the fiducial point of the monitored electrical activity using the RBB electrode is an earliest monitored QRS deflection.

Example Ex10: The implantable medical device or the method as in Example Ex9, wherein the respective earliest monitored QRS deflection is determined as the point where the respective monitored electrical activity crosses a respective isoelectric line of the monitored electrical activity using the LBB electrode, and of the monitored electrical activity using the RBB electrode, respectively.

Example Ex11: The implantable medical device or the method as in any one of Examples Ex1-Ex10, wherein to determine whether there is a cardiac physiological condition based on the determined difference, the computing apparatus is further configured to execute, or the method further comprises:
determining whether there is atrioventricular (AV) block based on a time from at least one of an atrial pacing event and an atrial sensing event to LV depolarization and a time from the at least one of the atrial pacing event and the atrial sensing event to RV depolarization.

Example Ex12: The implantable medical device or the method as Example Ex11, wherein determining whether there is AV block based on the time from the at least one of the atrial pacing event and the atrial sensing event to LV depolarization and the time from the at least one of the atrial pacing event and the atrial sensing event to RV depolarization comprises determining that the time from the at least one of the atrial pacing event and the atrial sensing event to LV depolarization and the time from the at least one of the atrial pacing event and the atrial sensing event to RV depolarization are above an AV conduction threshold.

Example Ex13: The implantable medical device or the method as in Example Ex12, wherein the AV conduction threshold is determined from one or more of electrical activity of a template heartbeat and a preset value.

Example Ex14: The implantable medical device or the method as in any one of Examples Ex1-Ex13, wherein to determine whether there is a cardiac physiological condition based on the determined difference, the computing apparatus is further configured to execute, or the method further comprises:
determining whether there is LBB block based on the determined difference.

Example Ex15: The implantable medical device or the method as in Example Ex14, wherein determining whether there is LBB block based on the determined difference comprises determining that the determined difference from LV depolarization to RV depolarization is negative.

Example Ex16: The implantable medical device or the method as in any one of Examples Ex1-Ex15, wherein to determine whether there is a cardiac physiological condition based on the determined difference, the computing apparatus is further configured to execute, or the method further comprises:
determining whether there is RBB block based on the determined difference.

Example Ex17: The implantable medical device or the method as in Example Ex16, wherein determining whether there is RBB block based on the determined difference comprises determining that the determined difference from LV depolarization to RV depolarization is positive and is above an RBB threshold.

Example Ex18: The implantable medical device or the method as in Example Ex17, wherein the RBB threshold is determined from one or more of electrical activity of a template heartbeat, and a preset value.

Example Ex19: The implantable medical device or the method as in any one of Examples Ex1-Ex18, wherein to determine whether there is a cardiac physiological condition based on the determined difference the computing apparatus is further configured to execute, or the method further comprises:
determining whether there is unblocked intact intrinsic AV conduction based on the determined difference.

Example Ex20: The implantable medical device or the method as in Example Ex19, wherein determining whether there is unblocked intact intrinsic AV conduction based on the determined difference comprises determining that the determined difference from LV depolarization to RV depolarization is positive and is below a normal conduction threshold.

Example Ex21: The implantable medical device or the method as in Example Ex20, wherein the normal conduction threshold is determined from one or more of electrical activity of a template heartbeat, and a preset value.

Example Ex22: A system comprising:
a conduction system pacing lead comprising:
one or more implantable electrodes comprising a left bundle branch (LBB) electrode positionable adjacent a portion of a patient's LBB and a right bundle branch (RBB) electrode positionable adjacent a portion of the patient's RBB; and
an implantable medical device comprising:
a computing apparatus comprising processing circuitry and operably coupled to the one or more implantable electrodes, wherein the computing apparatus is configured to:
monitor electrical activity using the one or more implantable electrodes;
detect electrical activity based on the monitored electrical activity indicative of intrinsic left ventricle (LV) depolarization and indicative of intrinsic right ventricle (RV) depolarization;
determine a difference based on the detected electrical activity indicative of intrinsic LV depolarization and the detected electrical activity indicative of intrinsic RV depolarization; and
determine whether there is a cardiac physiological condition based on the determined difference.

Example Ex 23: The system as in Example Ex 22, wherein the computing apparatus is further configured to:
select a pacing configuration based on the determined cardiac physiological condition; and
deliver pacing based on the selected pacing configuration using the one or more implanted electrodes.

Example Ex24: The system as in any one of Examples Ex22-Ex23, further comprising:
a ventricular lead comprising one or more implantable electrodes comprising a LV electrode positionable adjacent a portion of the patient's LV,
wherein the computing apparatus is further configured to detect electrical activity using the LV electrode indicative of intrinsic LV depolarization.

Example Ex25: The system as in any one of Examples Ex22-Ex24, further comprising:
an atrial lead comprising one or more implantable electrodes comprising a right atrium (RA) electrode positionable adjacent a portion of the patient's RA,
wherein the computing apparatus is further configured to detect intrinsic RA cardiac electrical activity or deliver RA pacing using the RA electrode.

This disclosure has been provided with reference to illustrative embodiments and examples and is not meant to be construed in a limiting sense. As described previously, one skilled in the art will recognize that other various illustrative applications may use the techniques as described herein to take advantage of the beneficial characteristics of the devices and methods described herein. Various modifications of the illustrative embodiments and examples will be apparent upon reference to this description.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

All references and publications cited herein are expressly incorporated herein by reference in their entirety for all purposes, except to the extent any aspect directly contradicts this disclosure.

All scientific and technical terms used herein have meanings commonly used in the art unless otherwise specified. The definitions provided herein are to facilitate understanding of certain terms used frequently herein and are not meant to limit the scope of the present disclosure.

Unless otherwise indicated, all numbers expressing feature sizes, amounts, and physical properties used in the specification and claims may be understood as being modified either by the term “exactly” or “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein or, for example, within typical ranges of experimental error.

The recitation of numerical ranges by endpoints includes all numbers subsumed within that range (e.g., 1 to 5 includes 1, 1.5, 2, 2.75, 3, 3.80, 4, and 5) and any range within that range. Herein, the terms “up to” or “no greater than” a number (e.g., up to 50) includes the number (e.g., 50), and the term “no less than” a number (e.g., no less than 5) includes the number (e.g., 5).

The terms “coupled” or “connected” refer to elements being attached to each other either directly (in direct contact with each other) or indirectly (having one or more elements between and attaching the two elements). Either term may be modified by “operatively” and “operably,” which may be used interchangeably, to describe that the coupling or connection is configured to allow the components to interact to carry out at least some functionality (for example, a mobile user device may be operatively coupled to a cellular network transmit data to or receive data therefrom).

Reference to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout are not necessarily referring to the same embodiment of the disclosure. Furthermore, the particular features, configurations, compositions, or characteristics may be combined in any suitable manner in one or more embodiments.

As used in this specification and the appended claims, the singular forms “a,” “an,” and “the” encompass embodiments having plural referents, unless the content clearly dictates otherwise. As used in this specification and the appended claims, the term “or” is generally employed in its sense including “and/or” unless the content clearly dictates otherwise.

As used herein, “have,” “having,” “include,” “including,” “comprise,” “comprising” or the like are used in their open-ended sense, and generally mean “including, but not limited to.” It will be understood that “consisting essentially of,” “consisting of,” and the like are subsumed in “comprising,” and the like.

The term “and/or” means one or all of the listed elements or a combination of at least two of the listed elements.

The phrases “at least one of,” “comprises at least one of,” and “one or more of” followed by a list refers to any one of the items in the list and any combination of two or more items in the list.

1. An implantable medical device comprising:
a computing apparatus comprising processing circuitry and operably coupled to one or more implantable electrodes comprising a left bundle branch (LBB) electrode positionable adjacent a portion of a patient's LBB, and a right bundle branch (RBB) electrode positionable adjacent a portion of the patient's RBB, wherein the computing apparatus is configured to:
monitor electrical activity using the LBB electrode and the RBB electrode;
detect electrical activity based on the monitored electrical activity indicative of intrinsic left ventricle (LV) depolarization and indicative of intrinsic right ventricle (RV) depolarization;
determine a temporal difference based on the detected electrical activity indicative of intrinsic LV depolarization and the detected electrical activity indicative of intrinsic RV depolarization; and
determine a cardiac physiological condition based on the determined difference.

2. (canceled)

3. The implantable medical device as in claim 1, wherein detecting electrical activity indicative of intrinsic LV depolarization is based on the monitored electrical activity using the LBB electrode, and wherein detecting electrical activity indicative of intrinsic RV depolarization is based on the monitored electrical activity using the RBB electrode.

4. The implantable medical device as in claim 1, wherein the one or more implantable electrodes further comprise an LV electrode positionable adjacent a portion of the patient's LV, and wherein detecting electrical activity indicative of intrinsic LV depolarization is based on the monitored electrical activity using the LV electrode.

5. The implantable medical device as in claim 1, wherein the one or more implantable electrodes further comprise a right atrium (RA) electrode positionable adjacent a portion of the patient's RA, and wherein the computing apparatus is further configured to
detect intrinsic RA cardiac electrical activity or delivering RA pacing using the RA electrode.

6. The implantable medical device as in claim 1, wherein the monitored electrical activity is based on at least one of the patient's intrinsic RA cardiac electrical activity and RA paced cardiac electrical activity, and wherein the detected electrical activity indicative of LV depolarization and indicative of RV depolarization is determined based on at least one of an initial monitored heartbeat or a second or subsequent monitored heartbeat.

7. The implantable medical device as in claim 1, wherein the computing apparatus is further configured to
select a pacing configuration based on the determined cardiac physiological condition; and
deliver pacing based on the selected pacing configuration using the one or more implanted electrodes.

8. The implantable medical device as in claim 1, wherein the determined difference based on the detected electrical activity indicative of LV depolarization and the detected electrical activity indicative of RV depolarization is a time period extending from a fiducial point of the monitored electrical activity using the LBB electrode to a fiducial point of the monitored electrical activity using the RBB electrode.

9. The implantable medical device as in claim 8, wherein the fiducial point of the monitored electrical activity using the LBB electrode is an earliest monitored QRS deflection and wherein the fiducial point of the monitored electrical activity using the RBB electrode is an earliest monitored QRS deflection.

10. (canceled)

11. The implantable medical device as in claim 1, wherein to determine the cardiac physiological condition based on the determined difference, the computing apparatus is further configured to
determine an atrioventricular (AV) block based on a time from at least one of an atrial pacing event and an atrial sensing event to LV depolarization and a time from the at least one of the atrial pacing event and the atrial sensing event to RV depolarization.

12. The implantable medical device as in claim 11, wherein determining the AV block based on the time from the at least one of the atrial pacing event and the atrial sensing event to LV depolarization and the time from the at least one of the atrial pacing event and the atrial sensing event to RV depolarization comprises determining that the time from the at least one of the atrial pacing event and the atrial sensing event to LV depolarization and the time from the at least one of atrial pacing event and the atrial sensing event to RV depolarization are above an AV conduction threshold.

13. (canceled)

14. The implantable medical device as in claim 1, wherein to determine the cardiac physiological condition based on the determined difference, the computing apparatus is further configured to
determine a LBB block based on the determined difference.

15. The implantable medical device as in claim 14, wherein determining the LBB block based on the determined difference comprises determining that the determined difference from LV depolarization to RV depolarization is negative.

16. The implantable medical device as in claim 1, wherein to determine the cardiac physiological condition based on the determined difference, the computing apparatus is further configured to
determine a RBB block based on the determined difference.

17. The implantable medical device as in claim 16, wherein determining the RBB block based on the determined difference comprises determining that the determined difference from LV depolarization to RV depolarization is positive and is above an RBB threshold.

18. (canceled)

19. The implantable medical device as in claim 1, wherein to determine the cardiac physiological condition based on the determined difference, the computing apparatus is further configured to
determine an unblocked intact intrinsic AV conduction based on the determined difference.

20. The implantable medical device as in claim 19, wherein determining the unblocked intact intrinsic AV conduction based on the determined difference comprises determining that the determined difference from LV depolarization to RV depolarization is positive and is below a normal conduction threshold.

21. (canceled)

22. A system comprising:
A conduction system pacing lead comprising:
one or more implantable electrodes comprising a left bundle branch (LBB) electrode positionable adjacent a portion of a patient's LBB and a right bundle branch (RBB) electrode positionable adjacent a portion of the patient's RBB; and
an implantable medical device comprising:
a computing apparatus comprising processing circuitry and operably coupled to the one or more implantable electrodes, wherein the computing apparatus is configured to:
monitor electrical activity using the one or more implantable electrodes;
detect electrical activity based on the monitored electrical activity indicative of intrinsic left ventricle (LV) depolarization and indicative of intrinsic right ventricle (RV) depolarization;
determine a difference based on the detected electrical activity indicative of intrinsic LV depolarization and the detected electrical activity indicative of intrinsic RV depolarization; and
determine a cardiac physiological condition based on the determined difference.

23. The system as in claim 22, wherein the computing apparatus is further configured to:
select a pacing configuration based on the determined cardiac physiological condition; and
deliver pacing based on the selected pacing configuration using the one or more implanted electrodes.

24. The system as in claim 22, further comprising:
a ventricular lead comprising one or more implantable electrodes comprising a LV electrode positionable adjacent a portion of the patient's LV,
wherein the computing apparatus is further configured to detect electrical activity using the LV electrode indicative of intrinsic LV depolarization.

25. The system as in claim 22, further comprising:
an atrial lead comprising one or more implantable electrodes comprising a right atrium (RA) electrode positionable adjacent a portion of the patient's RA,
wherein the computing apparatus is further configured to detect intrinsic RA cardiac electrical activity or deliver RA pacing using the RA electrode.

26. A method comprising:
monitoring electrical activity using one or more implantable electrodes comprising a left bundle branch (LBB) electrode positioned adjacent a portion of a patient's LBB and using a right bundle branch (RBB) electrode positioned adjacent a portion of the patient's RBB;
detecting electrical activity based on the monitored electrical activity indicative of intrinsic left ventricle (LV) depolarization and indicative of intrinsic right ventricle (RV) depolarization;
determining a difference based on the detected electrical activity indicative of intrinsic LV depolarization and the detected electrical activity indicative of intrinsic RV depolarization; and
determining a cardiac physiological condition based on the determined difference.

27. The method as in claim 26, wherein determining the cardiac physiological condition based on the determined difference comprises one or more of:
determining an unblocked intact intrinsic AV conduction based on the determined difference;
determining a LBB block based on the determined difference;
determining a RBB block based on the determined difference; and
determining an unblocked intact intrinsic AV conduction based on the determined difference.

28. The method as in claim 26, wherein the monitored electrical activity is based on at least one of the patient's intrinsic RA cardiac electrical activity and RA paced cardiac electrical activity, and wherein the detected electrical activity indicative of LV depolarization and indicative of RV depolarization is determined based on at least one of an initial monitored heartbeat or a second or subsequent monitored heartbeat.

29. The method as in claim 26, the method further comprising:
selecting a pacing configuration based on the determined cardiac physiological condition; and
delivering pacing based on the selected pacing configuration using the one or more implanted electrodes.

30. The method as in claim 26, wherein the determined difference based on the detected electrical activity indicative of LV depolarization and the detected electrical activity indicative of RV depolarization is a time period extending from a fiducial point of the monitored electrical activity using the LBB electrode to a fiducial point of the monitored electrical activity using the RBB electrode.
